(19) United States
(12) Patent Application Publication
(10) Pub. No.: US 2026/0365934 A1
(43) Pub. Date: Nov. 19, 2026

(54) Method for Producing High Calorific Value Fuel Gas

(71) Applicant: Osaka Gas Co., Ltd., Osaka-shi (JP)

(72) Inventors: Hirofumi Ohtsuka, Osaka-shi (JP); Shimpei Norioka, Osaka-shi (JP)

(21) Appl. No.: 19/130,187

(22) PCT Filed: Nov. 16, 2022
(86) PCT No.: PCT/JP2022/042576

§ 371 Date: May 15, 2025

(51) Int. Cl. C10L 3/08

(52) U.S. Cl. CPC C10L 3/08

(57) ABSTRACT

Provided is a method for producing a high calorific value fuel gas. The method includes a steam reforming process of bringing ethanol into contact with a catalyst containing ruthenium or nickel in the presence of hydrogen in an amount such that a molar ratio of hydrogen/ethanol is 1.0 or more and 10 or less and water vapor in an amount such that a molar ratio of water vapor/ethanol is 1.2 or more and 5 or less, under a condition where an inlet temperature of the catalyst is 200 °C or more and less than 400 °C; and a methanation process of bringing a gas obtained in the steam reforming process into contact with a catalyst containing ruthenium or nickel under a condition where an outlet temperature of the catalyst is 230 °C or more and 330 °C or less.

### CROSS-REFERENCE TO RELATED APPLICATION

This application is the United States national phase of International Patent Application No. PCT/JP2022/042576 filed Nov. 16, 2022, the disclosure of which is hereby incorporated by reference in its entirety.

### BACKGROUND OF THE INVENTION

### Field of the Invention

The present invention relates to a method for producing a high calorific value fuel gas that contains methane as the main component thereof, by methanation of ethanol in the presence of hydrogen.

### Description of Related Art

City gas, which contains natural gas as the main component thereof, is an environmentally friendly energy that emits less carbon dioxide during combustion than other fossil fuels such as petroleum, but in recent years in which environmental problems such as global warming have been becoming more serious, further reduction of carbon dioxide emissions is desired.

In recent years, biomass fuels such as bioethanol have been attracting attention as a new energy resource. Bioethanol is obtained through alcohol fermentation of sugar obtained by saccharification of starch contained in sugarcane juice or grains such as corn. Although burning bioethanol produces carbon dioxide, the process of growing the raw material plants takes in the corresponding amount of carbon dioxide from the air, so it can be considered that bioethanol does not increase the concentration of carbon dioxide in the atmosphere when totaled from the plant growth process, and for this reason, bioethanol is considered as a carbon neutral fuel.

If a method for efficiently producing a fuel gas containing methane as the main component thereof from ethanol can be established, it will be possible to reduce carbon dioxide emissions, for example, by using bioethanol as a raw material for city gas.

JP S52-52902A discloses a method for producing fuel gas by catalytic cracking of aliphatic alcohols with 2 to 5 carbon atoms or a mixture of such alcohols and water in the presence of a ruthenium-based catalyst. According to JP S52-52902A, a catalytic cracking reaction is typically carried out at 400 °C to 700 °C and 2 to 10 kg/cm² (absolute pressure), and by catalytically cracking ethanol in the presence of water vapor, a fuel gas of 4200 to 5050 kcal/m³ that contains 32% to 44% hydrogen by volume, 29% to 40% methane by volume, and 16% to 23% carbon dioxide by volume was obtained.

JP S55-144093A discloses an ethanol gasification method in which ethanol is mixed with water in a predetermined mixing ratio and is passed through a nickel-based catalyst at 300 °C to 600 °C. According to the description, the produced gaseous mixture is cooled to 250 °C to 350 °C and then introduced into the catalytic methanation process to obtain a fuel gas with a high methane concentration that can be used as synthetic natural gas after carbon dioxide removal, but there is no specific description on the catalytic methanation process nor specific description on the fuel gas composition after the catalytic methanation process.

JP 2009-227588A discloses a methane-containing gas production method for producing a methane-containing gas from an ethanol-containing raw material that contains ethanol and water vapor. The method includes a reaction process of passing the ethanol-containing raw material once through a catalyst in which rhodium or ruthenium is supported on the surface of an inorganic oxide serving as a support, at a reaction temperature of 400 °C or more.

Even with the method described in JP 2009-227588A, the methane concentration in the resulting methane-containing gas is only about 55% and cannot be used directly as a raw material for city gas.

Natural gas is commonly used as a raw material for city gas, and contains methane, which is the main component thereof, and small amounts of ethane, propane, and butane. Natural gas typically does not contain hydrogen or carbon monoxide, and carbon dioxide is removed in the process of refining natural gas. In particular, city gas produced from a liquefied natural gas is substantially free of hydrogen, carbon monoxide, or carbon dioxide as they are completely removed in the process of liquefaction and refining.

If hydrogen, carbon monoxide, or carbon dioxide is contained in city gas, the following problems may be caused.

First, carbon monoxide is highly toxic, and may cause a poisoning accident if the gas leaks. The allowable concentration thereof is 200 ppm, and from a safety perspective, the concentration in the fuel gas is desirably no higher than this level, and is required to be no higher than 1000 ppm even if dilution with air is taken into account.

Second, carbon dioxide is not only nonflammable, but also has the function of suppressing combustion. Therefore, when carbon dioxide is mixed into the fuel gas at a high concentration, not only the efficiency of gas transportation through pipeline may decrease due to a decrease in the calorific value of the fuel gas, but also the efficiency of the combustion equipment may decrease.

Finally, hydrogen is a fuel gas, but the calorific value per unit volume thereof is only approximately one-third that of methane, which is the main component of city gas. Therefore, if hydrogen is mixed into a fuel gas that contains methane as the main component thereof, the calorific value per unit volume thereof decreases. Furthermore, it is known that hydrogen has a large effect on combustion equipment because of the high combustion rate thereof.

As described above, if hydrogen, carbon monoxide, or carbon dioxide is mixed into city gas, they have various effects at each stage of gas supply and consumption, and therefore quality criteria for the gas to be fed to a city gas pipeline network generally impose restrictions on the concentrations of hydrogen, carbon monoxide and carbon dioxide.

Regarding a pipeline network in which fuel filling stations for natural gas vehicles are provided, there is a known example in which the upper limit of the hydrogen concentration is 2% by volume (E. I. Koytsoumpa and S. Karellas, Renewable and Sustainable Energy Reviews, Vol. 94, 2018, p. 536). In the natural gas transportation pipelines in the U. S. and Canada, there are many examples in which the carbon dioxide concentration limits are set to 2 to 4% by volume (M. M. Foss and C. Head, Interstate natural gas-quality specifications & interchangeability, Center for energy economics, 2004). In addition, an example in which the hydrogen concentration is restricted to 4% or less by volume, the carbon dioxide concentration is restricted to 0.5% or less by volume, and the carbon monoxide concentration is restricted to 0.05% or less by volume (Biogas Purchasing Guidelines, Osaka Gas Co., Ltd., 2008), and an example in which the total concentration of methane and ethane is restricted to 93% or more by volume, and the total concentration of components other than hydrocarbons is restricted to 4% or less by volume (Biogas Purchasing Guidelines, Tokyo Gas Co., Ltd., 2008) are also known.

The production of a gas containing methane as the main component thereof by steam reforming of ethanol is considered to proceed due to the following reaction. That is, hydrogen and carbon monoxide are produced by a steam reforming reaction of ethanol (Formula 1). A portion of the carbon monoxide is transformed into hydrogen and carbon dioxide by a CO shift reaction (Formula 2), and the remainder reacts with hydrogen to produce methane (Formula 3).

C₂H₅OH+H₂O->2CO+4H₂ (Formula 1)

0.5CO+0.5H₂O->0.5CO₂+0.5H₂ (Formula 2)

1.5CO+4.5H₂->1.5CH₄+1.5H₂O (Formula 3)

The overall reaction formula is given as Formula 4, where 1.5 moles of methane and 0.5 moles of carbon dioxide are produced from 1 mole of ethanol.

C₂H₅OH->1.5CH4+0.5CO₂ (Formula 4)

In other words, when ethanol simply undergoes a methanation reaction in the presence of water vapor, the produced gas will be a mixture of 75% methane and 25% carbon dioxide even if the reaction proceeds ideally, and the produced gas cannot be used as a raw material for city gas unless the carbon dioxide is removed in some way.

A hot potassium carbonate absorption method, an amine absorption method, and the like are known as carbon dioxide removal methods, and there are examples in which facilities for such carbon dioxide removal are used in plants for producing alternative natural gas containing naphtha or the like as a raw material. However, all of these carbon dioxide removal methods are expensive in terms of facility and operating costs, causing the problem that the economics of fuel gas production is significantly reduced.

JP 2013-540674A discloses a method for producing hydrogen from ethanol, the method including a process of passing ethanol through a nickel-based catalyst at a molar ratio of hydrogen/ethanol of 0.1 to 1.0 and a molar ratio of water vapor/ethanol of 1.0 to 10.0 at 300 °C to 600 °C, and converting ethanol into a flow containing methane, CO, CO₂, and H₂.

Table 5 of JP 2013-540674A indicates that the conversion of ethanol was performed over a commercially-available methanation catalyst under conditions of a molar ratio of water vapor/ethanol of 2.8, a molar ratio of hydrogen/ethanol of 250, 400 °C, and a pressure of 1 atm, and at a reaction time of 15 minutes, the conversion rate was 90.1%, methane selectivity was 98.8%, and gasoline selectivity was 1.2%, thus showing high methane selectivity, but at a reaction time of 60 minutes, the conversion rate decreased to 42.1%. However, in the reaction at a molar ratio of hydrogen/ethanol of 250, the resulting gas contains hydrogen as the main component thereof and methane, so it cannot be used as a raw material for city gas.

Furthermore, Table 7 of JP 2013-540674A indicates that when hydrogen was not added in the conversion of ethanol over the commercially-available methanation catalyst under conditions of a molar ratio of water vapor/carbon of 3, 400 °C, and a pressure of 10 atm, an increase in pressure loss in the catalyst layer due to carbon deposition occurred within a short time, whereas no increase in pressure loss was observed at a molar ratio of hydrogen/ethanol of 0.6. Also in this case, the composition of the resulting gas is 27.2% to 27.5% methane and 72.3% to 72.8% hydrogen, which means that the gas contains hydrogen as the main component thereof and methane, so it cannot be used as a raw material for city gas.

JP 2006-82996A describes a method for selectively reforming ethanol in the presence of a catalyst to produce a hydrogen-enriched product, wherein ethanol is passed through a dehydration/hydrogenation reactor at a molar ratio of ethanol to hydrogen of 0.2 to 1 to dehydrate/hydrogenate ethanol to produce ethane, the produced ethane is adiabatically reformed to produce a flow containing methane, the produced methane-containing flow is steam reformed to produce a mixture containing hydrogen and carbon monoxide, followed by a water gas shift reaction to obtain a hydrogen-enriched product.

However, J P 2006-82996A only shows results of conversion of ethanol to ethane, and there is no specific description on the methane concentration in the methane-containing flow.

JP 2010-524824A describes a hydrogen production process comprising combining a first feedstream and a second feedstream to produce, in a pre-reforming reactor, a first product stream comprising CH₄ and H₂O, wherein the first feedstream comprises a mixture of H₂ and at least one selected from the group consisting of hydrocarbons having two or more carbon atoms and alcohols having two or more carbon atoms, said mixture having a hydrogen stoichiometric ratio (λ) of at least 0.1; and the second feedstream comprises steam; feeding the first product stream into a reforming reactor; and reacting the first product stream in the reforming reactor to produce a second product stream comprising CO and H₂.

However, JP 2010-524824A only shows the effect of hydrogen addition on the propane conversion rate when propane is used as hydrocarbon and undergoes a steam reforming reaction at 450 °C on an iridium catalyst, but does not specifically disclose the composition of the produced gas, especially the concentration of methane.

JP S46-22749A discloses a high-purity methane production method, wherein in a methane production method by hydrogenolysis of a higher hydrocarbon than ethane, when methane is produced by passing hydrocarbon feedstocks such as petroleum distillates such as LPG, naphtha, and kerosene, and hydrogen gas (90% to 150% of the theoretical hydrogen content required to produce methane by hydrogenation of the feedstock) through a highly active nickel solid catalyst layer under heating, a mixture of the feedstock and water vapor in the amount required to control the maximum temperature in the range from 400 °C to 600 °C is added to the catalyst layer, and the reaction heat is removed from the catalyst layer by indirect heat exchange to keep the outlet temperature of the catalyst layer at least 100 °C lower than the maximum temperature.

JP S46-22749A discloses an example in which hexane, light straight run naphtha, kerosene distillate, or butane is used as a higher hydrocarbon than ethane, but does not disclose the use of alcohols.

Also, according to JP S46-22749A, in the high-purity methane production method, methane is obtained directly from a higher hydrocarbon than ethane without steam reforming and methanation, and water vapor is added to control the heat generation by hydrogenolysis.

The heat of reaction for the hydrogenolysis of ethane (Formula 5) is 66 kJ per mole of ethane, while methane production from ethanol (Formula 6) involves a much greater heat generation of 156 kJ per mole of ethanol and breaking a carbon-oxygen bond, so the nature of the reaction is also different. Therefore, JP S46-22749A does not provide any suggestion for the production of methane from ethanol.

C₂H₆+H₂->2CH₄ (Formula 5)

C₂H₅OH+2H₂->2CH₄+H₂O (Formula 6)

As another problem in obtaining a gas containing methane as the main component thereof by steam reforming of ethanol, there is the problem of carbon deposition as described in JP 2009-227588A and JP 2013-540674A. JP 2009-227588A discloses that significant carbon deposition occurred when a nickel catalyst was used in steam reforming at a ratio of water/ethanol of 0.78 (by weight). JP 2013-540674A discloses that carbon deposition occurred within a short time when hydrogen was not added in the conversion of ethanol on the commercially-available methanation catalyst at a molar ratio of water vapor/carbon of 3 and at 400 °C. The molar ratio of water/ethanol is 2 in JP 2009-227588A, and 6 in JP 2013-540674A. Steam reforming of ethanol requires the addition of a large amount of water vapor to avoid carbon deposition. The reaction to produce 1.5 moles of methane and 0.5 moles of carbon dioxide from 1 mole of ethanol (Formula 4) is an exothermic reaction, but the generated heat is only 74 kJ (25 °C), and about 1.7 moles of water vapor can only be produced even using the generated heat as the heat of vaporization of water. Therefore, in order to stably obtain a gas containing methane as the main component thereof by steam reforming of ethanol, an external supply of water vapor is required, which is also economically disadvantageous.

### SUMMARY OF THE INVENTION

In view of the above problems, in order to obtain a gas containing methane as the main component thereof with a high calorific value that can be used as city gas through a methanation reaction of ethanol, the present invention aims to provide a method for producing a high calorific value fuel gas that is economically advantageous and makes carbon deposition unlikely to occur, in which a high calorific value fuel gas with a high methane concentration that can be used directly as a raw material for city gas is obtained without using costly carbon dioxide separation facilities.

The method for producing a high calorific value fuel gas according to the present invention has a feature configuration in which it includes: a steam reforming process of bringing ethanol into contact with a catalyst containing ruthenium or nickel in the presence of hydrogen in an amount such that a molar ratio of hydrogen/ethanol is 1.0 or more and 10 or less, and water vapor in an amount such that a molar ratio of water vapor/ethanol is 1.2 or more and 5 or less, under a condition where an inlet temperature of the catalyst is 200 °C or more and less than 400 °C; and
a methanation process of bringing a gas obtained in the steam reforming process into contact with a catalyst containing ruthenium or nickel under a condition where an outlet temperature of the catalyst is 230 °C or more and 330 °C or less.

According to this feature configuration, carbon dioxide, which is produced in a large amount as a byproduct in conventional fuel gas production methods that combine steam reforming of ethanol and a methanation reaction, reacts with hydrogen and is converted into methane. Therefore, it is possible to avoid large amount of carbon dioxide from remaining in the resulting fuel gas, and obtain a high calorific value fuel gas that can be used as a raw material for city gas without using costly carbon dioxide removal facilities, and carbon deposition is hardly to occur.

Furthermore, the steam reforming process can be started at a lower temperature than in a case where the steam reforming process of ethanol is performed in the absence of hydrogen, and the energy required to preheat the raw material gas in the steam reforming process can be saved, allowing a fuel gas with a higher calorific value to be produced more economically.

In addition, in the conventional fuel gas production method that combines steam reforming of ethanol with a methanation reaction, only 1.5 moles of methane per mole of ethanol are produced, whereas according to this feature configuration, approximately 2 moles of methane per mole of ethanol can be obtained, allowing a fuel gas with a higher calorific value to be produced more economically.

A further feature configuration of the method for producing a high calorific value fuel gas according to the present invention is such that a raw material gas to be subjected to the steam reforming process further contains at least one of carbon monoxide and carbon dioxide.

According to this feature configuration, the steam reforming process can be started at a lower temperature because the methanation reactions of carbon monoxide and carbon dioxide, which proceed rapidly even at low temperatures of about 200 °C to 250 °C and generate somewhat greater heat, proceeds in advance and thus the temperature rises rapidly in the steam reforming catalyst layer. With this, the energy required to preheat the raw material gas in the steam reforming process can be saved, allowing a fuel gas with a higher calorific value to be produced more economically.

A further feature configuration of the method for producing a high calorific value fuel gas according to the present invention is such that an amount of hydrogen to be subjected to the steam reforming process has a value such that a value of {(hydrogen)+(ethanol)×6+(carbon monoxide)}/{(ethanol)×2+(carbon monoxide)+(carbon dioxide)} calculated based on an amount of substance (mol) is 3.9 or more and 4.1 or less.

According to this feature configuration, a high calorific value fuel gas with high methane purity can be obtained that can suppress residual hydrogen and carbon dioxide in the resulting fuel gas at a low level, and is easy to use as a raw material for city gas.

A further feature configuration of the method for producing a high calorific value fuel gas according to the present invention is such that it further includes: a process of conditioning a raw material gas by adding hydrogen and water vapor to ethanol so that the molar ratio of hydrogen/ethanol is 1.8 or more and 2.2 or less and the molar ratio of water vapor/ethanol is 1.2 or more and 4 or less, wherein the raw material gas is subjected to the steam reforming process.

According to this feature configuration, a high calorific value fuel gas with high methane purity can be obtained that can suppress residual hydrogen and carbon dioxide in the resulting fuel gas at a low level, and is easy to use as a raw material for city gas. In addition, since carbon deposition onto the catalyst is difficult to occur, a fuel gas can be produced stably over a long period of time.

A further feature configuration of the method for producing a high calorific value fuel gas according to the present invention is such that it further includes: a process of conditioning a raw material gas by adding hydrogen and water vapor to ethanol so that the molar ratio of hydrogen/ethanol is 1.0 or more and less than 1.8 and the molar ratio of water vapor/ethanol is 2 or more and 4 or less, wherein the raw material gas is subjected to the steam reforming process, hydrogen is added to a gas obtained in the steam reforming process, and a resulting gas is subjected to the methanation process.

According to this feature configuration, the peak temperature of the steam reforming catalyst layer can be suppressed even when the steam reforming process is performed under adiabatic conditions, thus suppressing thermal degradation of the catalyst. As a means for suppressing thermal degradation of the catalyst in the exothermic reaction, a method of diluting a raw material gas by a recycling operation, in which a portion of the gas after the reaction is returned to the reactor inlet, is employed. But, by employing this feature configuration, the steam reforming process can be performed without suppressing the recycle rate or performing the recycle operation. Therefore, it is possible to obtain a high calorific value fuel gas with high methane purity more economically.

A further feature configuration of the method for producing a high calorific value fuel gas according to the present invention is such that the raw material gas conditioned by adding hydrogen and water vapor to ethanol is mixed with a portion of a gas obtained in the steam reforming process, and is then subjected to the steam reforming process.

According to this feature configuration, the peak temperature of the steam reforming catalyst layer can be suppressed even when the steam reforming process is performed under adiabatic conditions, thus suppressing thermal degradation of the catalyst.

### BRIEF DESCRIPTION OF THE DRAWINGS

FIG. 1 is a block flow diagram showing an aspect of a method for producing a high calorific value fuel gas according to the present invention.

FIG. 2 is a block flow diagram showing another aspect of the method for producing a high calorific value fuel gas according to the present invention.

FIG. 3 is a block flow diagram showing yet another aspect of the method for producing a high calorific value fuel gas according to the present invention.

FIG. 4 is a block flow diagram showing yet another aspect of the method for producing a high calorific value fuel gas according to the present invention.

FIG. 5 is a block flow diagram showing yet another aspect of the method for producing a high calorific value fuel gas according to the present invention.

FIG. 6 is a diagram showing relationships between the temperature and the carbon activity in a catalyst layer at ratios of water vapor/ethanol (H2O/EtOH) in Examples 3 to 6 and Comparative Example 1.

FIG. 7 is a diagram showing relationships between the temperature and the carbon activity at ratios of water vapor/ethanol (H₂O/EtOH) in Comparative Examples 2 to 4.

FIG. 8 is a process flow diagram showing an aspect of the method for producing a high calorific value fuel gas according to the present invention.

FIG. 9 is a process flow diagram showing yet another aspect of the method for producing a high calorific value fuel gas according to the present invention.

FIG. 10 is a process flow diagram showing yet another aspect of the method for producing a high calorific value fuel gas according to the present invention.

### DESCRIPTION OF THE INVENTION

### Embodiment

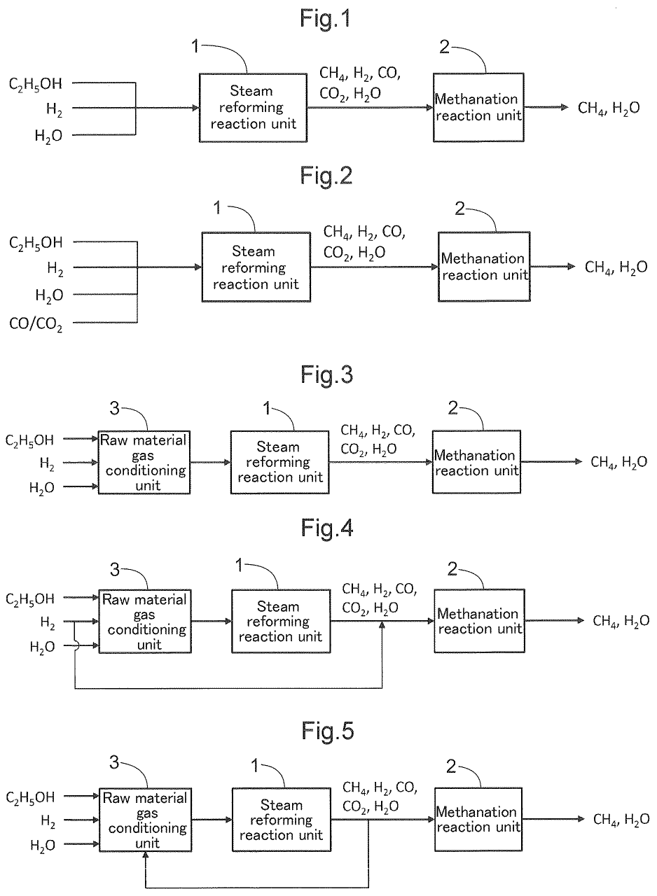

The following describes an embodiment of a method for producing a high calorific value fuel gas according to the present invention. FIG. 1 is a block flow diagram showing the method for producing a high calorific value fuel gas according to the present invention. The method for producing a high calorific value fuel gas includes: a steam reforming process of bringing ethanol into contact with a catalyst containing ruthenium or nickel in the presence of hydrogen in an amount such that a molar ratio of hydrogen/ethanol is 1.0 or more and 10 or less, and water vapor in an amount such that a molar ratio of water vapor/ethanol is 1.2 or more and 5 or less, under a condition where an inlet temperature of the catalyst is 200 °C or more and less than 400 °C (steam reforming reaction unit 1); and a methanation process of bringing the gas obtained in the steam reforming process into contact with a catalyst containing ruthenium or nickel under a condition where an outlet temperature of the catalyst is 230 °C or more and 330 °C or less (methanation reaction unit 2). According to the method for producing a high calorific value fuel gas having such configurations, it is possible to obtain a high calorific value fuel gas in which the ratio of methane is 85% or more by volume after desiccation, more preferably 90% or more, and even more preferably 95% or more.

Ethanol used in the method for producing a high calorific value fuel gas of the present invention does not need to be produced by a fermentation method, but is preferably bioethanol produced from sugarcane or corn, from the perspective of producing fuel gas that can be regarded as being carbon neutral.

Ethanol typically contains a small amount of water and may also contain extremely small amounts of organic acids, aldehydes, and sulfur compounds such as thiols. Of them, water may be contained in the range from about 20% to 50% of ethanol on a mass basis. If ethanol used as a raw material contains water, the amount of water vapor to be added can be adjusted according to the amount of water.

Extremely small amounts of organic acids and aldehydes may cause carbon deposition and the like in the subsequent steam reforming process if contained in too large amounts, but this is usually not a problem.

Sulfur compounds such as thiols may cause sulfur poisoning in the subsequent steam reforming process, resulting in a reduction in activity, so if a large amount of sulfur compounds are contained, it is desirable to perform a desulfurization treatment in advance.

Hydrogen produced by any method may be used as hydrogen used in the method for producing a high calorific value fuel gas of the present invention, but hydrogen produced using water electrolysis devices such as an alkaline electrolysis device, a solid polymer electrolysis device, and a solid oxide electrolysis device is preferable because it does not usually contain impurities that can interfere with the steam reforming process.

There are no particular restrictions on the method or order of mixing hydrogen and water vapor with ethanol, but the molar ratio of hydrogen/ethanol is preferably 1.0 or more and 10 or less. If the molar ratio of hydrogen/ethanol is less than 1.0, it will be difficult to reduce the carbon dioxide concentration in the fuel gas. If the molar ratio of hydrogen/ethanol is greater than 10, it will be difficult to reduce the hydrogen concentration in the fuel gas.

The molar ratio of water vapor/ethanol is preferably 1.2 or more and 5 or less, more preferably 1.5 or more and 4 or less, and furthermore preferably 2 or more and 3.5 or less. If the molar ratio of water vapor/ethanol is less than 1.2, it will be difficult to suppress carbon deposition on the catalyst. If the molar ratio of water vapor/ethanol is more than 5, it will be difficult to reduce the energy required for water vapor production, which is economically disadvantageous.

The method for producing a high calorific value fuel gas of the present invention includes a steam reforming process of bringing gas containing ethanol, hydrogen, and water vapor into contact with a catalyst containing ruthenium or nickel under a condition where the inlet temperature of the catalyst is 200 °C or more and less than 400 °C.

The steam reforming catalyst used in the steam reforming process is a catalyst with ruthenium or nickel supported on a support made of inorganic oxide such as alumina. A catalyst with ruthenium supported on a support made of inorganic oxide such as alumina is particularly preferable because it is less likely to cause carbon deposition. The steam reforming process may be performed in an adiabatic reactor, or may be performed using a heat exchanger reactor so that the catalyst layer temperature is kept nearly constant. The steam reforming catalyst is filled in the steam reforming reaction unit 1 to form a steam reforming catalyst layer.

The steam reforming process is performed under the condition where the inlet temperature of the steam reforming catalyst layer is 200 °C or more and less than 400 °C. In the steam reforming process, the steam reforming reaction of ethanol, which is an endothermic reaction, and the methanation reaction, which is an exothermic reaction, occur in conjunction with each other, resulting in an exothermic reaction as a whole. If using a reactor without a special heat removal mechanism (adiabatic reactor), a raw material gas is fed with the inlet temperature of the steam reforming catalyst layer set to about 300 °C, and the reaction is conducted under a substantially adiabatic condition, the outlet temperature of the steam reforming catalyst layer will typically be about 500 °C to 700 °C. As a result of diligent study by the inventors and others, it was found that the steam reforming reaction of ethanol proceeds in conjunction with an increase in temperature when the raw material gas is fed with the inlet temperature of the steam reforming catalyst layer set to about 250 °C, and that the steam reforming reaction of ethanol proceeds rapidly when the temperature of the steam reforming catalyst layer is 400 °C or more. Therefore, the reforming reaction of ethanol is likely to proceed completely when conducted in an adiabatic reactor.

Using a heat exchanger reactor, which is a reactor having a heat exchange function, allows the reaction to proceed while removing the heat generated by the reaction, so the steam reforming reaction can proceed while suppressing an increase in temperature of the catalyst layer, which is advantageous in terms of catalyst durability. When the reactor is properly designed and operated, the reaction can be conducted isothermally, with the heat generated by the reaction removed and the inlet temperature of the steam reforming catalyst layer nearly matching the outlet temperature of the steam reforming catalyst layer. However, it should be noted that even in a heat exchanger reactor, the temperature of the catalyst layer is distributed, and the temperature may rise to a temperature close to the outlet temperature of an adiabatic reaction at a location where an exothermic reaction is in progress.

The methanation catalyst used in the methanation process is a catalyst with ruthenium or nickel supported on a support made of inorganic oxide such as alumina. A catalyst with ruthenium supported on a support made of inorganic oxide such as alumina is particularly preferable because it is less likely to cause carbon deposition. The methanation process may be performed in an adiabatic reactor, or may be performed using a heat exchanger reactor so that the catalyst layer temperature is kept nearly constant. The methanation catalyst is filled in the methanation reaction unit 2 to form a methanation catalyst layer.

During the methanation process, a methanation reaction, which is an exothermic reaction, proceeds, and thus if the reaction is conducted in an adiabatic reactor, the reaction gas will flow out at a higher temperature than the inlet temperature of the methanation catalyst layer. Since the methanation reaction proceeds in equilibrium to the methane generation side at lower temperatures, it is advantageous to set a lower outlet temperature of the methanation catalyst layer from the viewpoint of obtaining a fuel gas with high methane purity, and thus the methanation reaction is conducted under a condition where the outlet temperature of the methanation catalyst layer is no higher than 330 °C. On the other hand, at lower temperatures, the activity of the methanation catalyst decreases, and it is difficult to allow the reaction to proceed at temperatures below 230 °C. Therefore, the outlet temperature of the methanation catalyst layer is preferably 230 °C or more and 330 °C or less, and more preferably 230 °C or more and 280 °C or less.

When the methanation reactor is composed of adiabatic reactors, multiple reactors are provided and coolers are provided between the reactors so that the inlet temperature of each reaction stage is 230 °C or more and 330 °C or less, and more preferably 230 °C or more and 280 °C or less, and the outlet temperature of the catalyst layer in the final stage reactor is 230 °C or more and 330 °C or less.

The pressure in the steam reforming reaction unit 1 in the steam reforming process is preferably 0.3 MPa (absolute pressure, the same hereinafter) or more and 5 MPa or less, and more preferably 0.5 MPa or more and 3 MPa or less, because a sufficient reaction rate is easily obtained when the pressure is 0.3 MPa or more, and the facility costs can be suppressed when the pressure is 5 MPa or less, which is economically advantageous.

The pressure in the methanation reaction unit 2 in the methanation process is preferably 0.3 MPa or more and 5 MPa or less, and more preferably 0.5 MPa or more and 3 MPa or less, because a sufficient reaction rate is easily obtained and methanation easily proceeds in equilibrium when the pressure is 0.3 MPa or more, and the facility costs can be suppressed when the pressure is 5 MPa or less, which is economically advantageous.

In both the steam reforming process and the methanation process, the pressure in the steam reforming reaction unit 1 and the pressure in the methanation reaction unit 2 are preferably 0.3 MPa or more and 5 MPa or less, and more preferably 0.5 MPa or more and 3 MPa or less, so it is typically not necessary to change the pressure between the processes, but a process of pressurization or depressurization in the steam reforming reaction unit 1 or the methanation reaction unit 2 may be added, if necessary. Typically, the reaction pressure decreases slightly in accordance with the pressure loss in each process, but there is no particular problem.

FIG. 2 is a block flow diagram showing yet another aspect of the method for producing a high calorific value fuel gas according to the present invention. In the method for producing a high calorific value fuel gas according to the present embodiment, the raw material gas to be subjected to the steam reforming process contains, in addition to ethanol, hydrogen, and water vapor, at least one of carbon monoxide and carbon dioxide.

Carbon dioxide may be carbon dioxide recovered from combustion exhaust gases or exhaust gases in chemical processes using a known carbon dioxide recovery method such as an amine absorption method, or may be recovered from the atmosphere.

Note that if carbon dioxide contains a sulfur compound, this may poison and degrade the steam reforming catalyst and the methanation catalyst, so a treatment of desulfurizing carbon dioxide is performed if necessary.

Carbon monoxide may be carbon monoxide synthesized by electrochemical reduction of carbon dioxide as described above, or may be carbon monoxide obtained by reduction through a reaction of carbon dioxide as described above with hydrogen (reverse water gas shift reaction).

In this aspect, if the amount of hydrogen to be subjected to the steam reforming process is added so that the value of {(hydrogen)+(ethanol)×6+(carbon monoxide)}/{(ethanol)×2+(carbon monoxide)+(carbon dioxide)} calculated based on an amount of substance (mol) is 3.9 or more and 4.1 or less, the fuel gas obtained after the methanation process has low concentrations of components other than methane, resulting in a fuel gas with high methane purity and a high calorific value. If the above value is smaller than 3.9, the amount of carbon dioxide remaining in the obtained fuel gas may be high. If the above value is greater than 4.1, the amount of hydrogen remaining in the obtained fuel gas may be high.

FIG. 3 is a block flow diagram showing yet another aspect of the method for producing a high calorific value fuel gas according to the present invention. The method for producing a high calorific value fuel gas according to the present embodiment includes, in addition to the basic aspect shown in FIG. 1, a raw material gas conditioning process of adding hydrogen and water vapor to ethanol in a raw material gas conditioning unit 3 to condition the raw material gas so that the molar ratio of hydrogen/ethanol is 1.8 or more and 2.2 or less and the molar ratio of water vapor/ethanol is 1.2 or more and 4 or less. The conditioned gas after the raw material gas conditioning process is configured to be subjected to the steam reforming process.

The molar ratio of hydrogen/ethanol to be fed to the raw material gas conditioning process is preferably 1.8 or more and 2.2 or less. With this configuration, it is possible to suppress the residual hydrogen and carbon dioxide in the obtained fuel gas to a particularly low level and obtain a fuel gas with high methane purity and a high calorific value that is likely to be used as a raw material for city gas. If the molar ratio of hydrogen/ethanol is less than 1.8, it may be difficult to reduce the carbon dioxide concentration in the fuel gas. If the molar ratio of hydrogen/ethanol is more than 2.2, it may be difficult to reduce the hydrogen concentration in the fuel gas.

The molar ratio of water vapor/ethanol that is fed to the raw material gas conditioning process is preferably 1.2 or more and 4 or less. The heat generated by a reaction to produce 2 moles of methane and 1 mole of water from 1 mole of ethanol and 2 moles of hydrogen is 156 kJ (25 °C), so if the heat as a whole is used as heat of vaporization of water, approximately 3.5 moles of water vapor can be produced. If the molar ratio of water vapor/ethanol is 1.5 or more, carbon deposition is likely to be avoided, while if the molar ratio of water vapor/ethanol is 3 or less, the required amount of water vapor is suppressed, which is economically advantageous. Thus, the molar ratio of water vapor/ethanol is more preferably 1.5 or more and 3 or less.

Note that, in order to suppress the outlet temperature of the steam reforming catalyst layer, methane may be added in the amount that corresponds to the molar ratio of methane/ethanol of 0.5 or more and 1.5 or less. In this case, in order to avoid carbon deposition, the molar ratio of water vapor/ethanol is preferably higher than the above-mentioned molar ratio, namely, 2.5 or more and 4 or less.

FIG. 4 is a block flow diagram showing yet another aspect of the method for producing a high calorific value fuel gas according to the present invention. The method for producing a high calorific value fuel gas according to the present embodiment includes, in addition to the basic aspect shown in FIG. 1, a raw material gas conditioning process of adding hydrogen and water vapor to ethanol in the raw material gas conditioning unit 3 to condition the raw material gas so that the molar ratio of hydrogen/ethanol is 1.0 or more and less than 1.8 and the molar ratio of water vapor/ethanol is 2 or more and 4 or less. A configuration is such that the conditioned gas after the raw material gas conditioning process is subjected to the steam reforming process, hydrogen is added to the outlet gas of the steam reforming reaction unit obtained in the steam reforming process, and the resulting gas is subjected to the methanation process.

The molar ratio of hydrogen/ethanol to be fed to the raw material gas conditioning process is 1.0 or more and less than 1.8. With this configuration, the peak temperature of the steam reforming catalyst layer is suppressed when the steam reforming process is performed under adiabatic conditions. If the molar ratio of hydrogen/ethanol is 1.5 or less, the suppression effect is greater. If the molar ratio of hydrogen/ethanol is 1.0 or less, the progress of the methanation reaction slows down due to the decrease in hydrogen concentration, and the temperature increase due to the progress of the methanation reaction is small, causing the risk that the reforming reaction of ethanol does not progress sufficiently. If the molar ratio of hydrogen/ethanol is 1.8 or more, the effect of suppressing the peak temperature of the steam reforming catalyst layer is poor. In the present specification, “peak temperature of the catalyst layer” refers to the maximum value of the temperature of the catalyst layer.

The molar ratio of water vapor/ethanol is preferably 2 or more and 4 or less, but if the molar ratio of water vapor/ethanol is 2.5 or more, carbon deposition of the catalyst is likely to be avoided, whereas if the molar ratio of water vapor/ethanol is 3 or less, the required amount of water vapor is suppressed, which is economically advantageous. Thus, the molar ratio of water vapor/ethanol is preferably 2.5 or more and 3 or less. If the molar ratio of water vapor/ethanol is less than 2.5, the outlet temperature of the steam reforming reaction unit will be higher and the carbon activity will exceed 1, which may cause degradation of the steam reforming catalyst due to thermal degradation and carbon deposition. If the molar ratio of water vapor/ethanol is more than 4, a large amount of water vapor is required, which may deteriorate economic efficiency.

In the present embodiment, hydrogen is added to the outlet gas of the steam reforming reaction unit obtained in the steam reforming process, and the resulting gas is subjected to the methanation process. The additive amount of hydrogen is preferable if the molar ratio of hydrogen in total with the hydrogen mixed with ethanol in the raw material gas conditioning process to ethanol is 1.8 or more and 2.2 or less, because it is easy to obtain a fuel gas with high methane purity and a high calorific value, and is more preferable if this molar ratio is 2.0 or more and 2.1 or less.

FIG. 5 is a block flow diagram showing yet another aspect of the method for producing a high calorific value fuel gas according to the present invention. The method for producing a high calorific value fuel gas according to the present embodiment has, in addition to the basic aspect shown in FIG. 3, a configuration in which the conditioned gas conditioned in the raw material gas conditioning process and a portion of the outlet gas of the steam reforming reaction unit obtained in the steam reforming process are mixed, and then the mixture is subjected to the steam reforming process.

The steam reforming process is an exothermic reaction, and the steam reforming catalyst is exposed to high temperatures, which may cause a durability problem. In this aspect, a portion of the outlet gas of the steam reforming reaction unit is mixed with the conditioned gas, and the conditioned gas is diluted, thereby mitigating the temperature increase in the steam reforming process.

The raw material gas conditioning unit 3 may be provided with a recycling compressor 5 for recycling a portion of the outlet gas of the steam reforming reaction unit 1 (outlet gas of the steam reforming reaction unit) and a flow control means for controlling the recycling amount.

### EXAMPLES AND COMPARATIVE EXAMPLES

The following Test Examples 1 to 4 are test examples regarding the steam reforming process in the method for producing a high calorific value fuel gas of the present invention.

### Test Example 1

A sheath tube (outer diameter 6 mm) for temperature measurement was passed through the center of a stainless steel reaction tube (inner diameter 20 mm), and 12 mL (about 9 g) of catalyst with 2 mass % ruthenium supported on active alumina supports (spherical shape of 2 to 4 mm) was filled between the reaction tube and the sheath tube to form a catalyst layer. Both above and below the catalyst layer, alumina balls were filled to a height of 30 mm. While the reaction tube was loaded in an electric furnace and heated so that the temperature at the center of the alumina ball layer on the catalyst layer was 250 °C, a reduction treatment was performed for 1 hour by passing a reducing gas that is a mixture of nitrogen gas and 2% (by volume) hydrogen gas.

After the above-described reduction treatment, while the pressure in the reaction tube was maintained at 0.7 MPa (absolute pressure) and heating was performed so that the temperature of the center of the alumina ball layer on the catalyst layer, i.e., 15 mm above the upper end of the catalyst layer, was 250 °C, a raw material gas that is a mixture of ethanol at 200 mL/min (volume under the standard condition of 0 °C and 1 atm, the same hereinafter), hydrogen at 400 mL/min, and water vapor at 400 mL/min was passed through the inlet of the catalyst layer, from the top to the bottom. From the outlet gas of the catalyst layer, the condensed components (water and ethanol) were separated in an ice-cold trap, and then carbon monoxide, carbon dioxide, hydrogen, and nitrogen in the gas were analyzed using a gas chromatograph (Micro-GC with TCD detector manufactured by Agilent Inc.), and hydrocarbons (methane, ethylene, ethane, propylene, propane, and butane) and ethanol were analyzed using a gas chromatograph (GC-2014 with FID detector manufactured by Shimadzu Corporation). Also, ethanol in the condensate condensed in the ice-cold trap was analyzed by the gas chromatograph (GC-2014 with FID detector manufactured by Shimadzu Corporation).

The reason why the temperature control point was set in a portion of the alumina ball layer above the upper end of the catalyst layer, rather than inside the catalyst layer, was that it was difficult to accurately measure the temperature inside the catalyst layer due to heat generated by the reaction. Because the reaction does not proceed within the alumina ball layer, the temperature of the gas at the inlet of the catalyst layer substantially corresponds to that at the center of the alumina ball layer. In the following, the inlet temperature of the catalyst layer refers to the temperature at the center of the alumina ball layer on the catalyst layer. Note that the temperatures inside the catalyst layer (at three portions, namely, top, middle, and bottom) were also measured using a thermocouple inserted in the sheath tube.

After the measurements at 250 °C were completed, the inlet temperature of the catalyst layer was successively changed to 300 °C, 350 °C, and 400 °C while passing the test gas, and the outlet gas of the catalyst layer was analyzed using a gas chromatograph in the same manner.

The ethanol conversion rate in the reaction was calculated using the following formula. The ethanol contained in the outlet gas of the catalyst layer contains, in addition to the gas that passed through the ice-cold trap, ethanol dissolved in the water condensed in the ice-cold trap.

Ethanol conversion rate [%]=100×{1−(amount of substance per unit time of ethanol contained in the outlet gas of the catalyst layer)/(amount of substance per unit time of ethanol fed to the catalyst layer)}

Table 1 shows, for each inlet temperature of the catalyst layer, temperatures of the top, middle, and bottom of the catalyst layer, the gas composition analysis after condensate separation, and the ethanol conversion rate.

When the inlet temperature of the catalyst layer was 250 °C, the produced gas was a methane-dominant gas that contained 88.7% methane, 9.3% hydrogen, and 2.0% carbon dioxide. The ethanol conversion rate was 98.0%, and some ethanol remained in the condensate. The maximum temperature inside the catalyst layer was 339 °C (top) and 284 °C at the outlet of the catalyst layer. Although the temperature inside the catalyst layer was higher than the temperature at the inlet of the catalyst layer due to the heat generated by methane production, it can be seen that the catalyst layer was not sufficiently insulated and thus the temperature decreased due to heat dissipation inside the catalyst layer.

When the inlet temperature of the catalyst layer was 300 °C, the produced gas was a methane-dominant gas that contains 87.6% methane. The ethanol conversion rate was 100%, and no ethanol was detected both in the gas and in the condensate. The maximum temperature inside the catalyst layer was 371 °C. Since the maximum temperature inside the catalyst layer was higher than when the inlet temperature of the catalyst layer was 250 °C, it was conceivable that complete ethanol conversion was achieved.

When the inlet temperature of the catalyst layer was 350 °C and 400 °C, due to an increase in outlet temperature of the catalyst layer, the methane concentration of the produced gas was slightly lower than when the inlet temperature of the catalyst layer was 300 °C.

TABLE 1

| | | | | | | |
|---|---|---|---|---|---|---|
| Catalyst | Inlet | °C | 250 | 300 | 350 | 400 |
| temperature | Top | °C | 339 | 371 | 408 | 445 |
| | Middle | °C | 315 | 347 | 384 | 422 |
| | Bottom | °C | 284 | 319 | 357 | 397 |
| Gas concentration | H₂ | vol % | 9.3 | 10.2 | 14.2 | 19.4 |
| | CO | vol % | 0.0 | 0.0 | 0.0 | 0.1 |
| | CO₂ | vol % | 2.0 | 2.2 | 3.2 | 4.5 |
| | CH₄ | vol % | 88.7 | 87.6 | 82.6 | 76.0 |

| | | | | | |
|---|---|---|---|---|---|
| Ethanol conversion rate | % | 98.0 | 100.0 | 100.0 | 100.0 |

### Test Example 2

The test was conducted as in Test Example 1, except that half of the hydrogen in the raw material gas supplied to the catalyst layer was replaced with nitrogen so that the gas contained ethanol at 200 mL/min, hydrogen at 200 mL/min, nitrogen at 200 mL/min, and water vapor at 400 mL/min. Nitrogen was added to make the total flow rate of the gas supplied to the catalyst per unit time equal to that in Test Example 1.

Table 2 shows, for each inlet temperature of the catalyst layer, temperatures of the top, middle, and bottom of the catalyst layer, the gas composition analysis after condensate separation, and the ethanol conversion rate.

When the inlet temperature of the catalyst layer was 250 °C, the produced gas was a methane-dominant gas that contains 51.6% methane, 5.9% hydrogen, 8.7% carbon dioxide, and extremely small amounts (0.02% or less) of ethane and propane. Even after nitrogen was removed, a large amount of carbon dioxide remained in the produced gas because, stoichiometrically, there was not enough hydrogen to methanate all the carbons contained in ethanol. The ethanol conversion rate was 97.8%, and some ethanol remained in the gas, in addition to the condensate. The maximum temperature inside the catalyst layer was 363 °C (at the top and middle) and 336 °C in the bottom of the catalyst layer. When the inlet temperature of the catalyst layer was 300 °C or more, the ethanol conversion rate was 100% and carbon hydride in the produced gas was only methane.

TABLE 2

| | | | | | | |
|---|---|---|---|---|---|---|
| Catalyst | Inlet | °C | 250 | 300 | 350 | 400 |
| temperature | Top | °C | 363 | 387 | 422 | 459 |
| | Middle | °C | 363 | 376 | 410 | 447 |
| | Bottom | °C | 336 | 353 | 387 | 426 |
| Gas concentration | H₂ | vol % | 5.9 | 6.0 | 8.8 | 12.6 |
| | CO | vol % | 0.1 | 0.0 | 0.1 | 0.2 |
| | CO₂ | vol % | 8.7 | 8.8 | 9.2 | 9.8 |
| | CH₄ | vol % | 51.6 | 51.6 | 49.4 | 46.3 |
| | N₂ | vol % | 33.4 | 33.2 | 32.1 | 30.7 |

| | | | | | |
|---|---|---|---|---|---|
| Ethanol conversion rate | % | 97.8 | 100.0 | 100.0 | 100.0 |

### Test Example 3

The test was conducted as in Test Example 1, except that the total amount of the hydrogen in the gas supplied to the catalyst layer was replaced with nitrogen so that the gas contained ethanol at 200 mL/min, nitrogen at 400 mL/min, and water vapor at 400 mL/min.

Table 3 shows, for each inlet temperature of the catalyst layer, temperatures of the top, middle, and bottom of the catalyst layer, the gas composition analysis after condensate separation, and the ethanol conversion rate.

When the inlet temperature of the catalyst layer was 250 °C, the ethanol conversion rate was only 11.5%. The ethanol conversion rate increased as the inlet temperature of the catalyst layer increased, but the ethanol conversion rate remained 64.6% even when the inlet temperature of the catalyst layer was 400 °C. At all the temperatures, the produced gas (after excretion of nitrogen) contained hydrogen as the main component thereof, and contained carbon monoxide, carbon dioxide, methane, and small amounts (0.02% to 4.0%) of C2 to C4 hydrocarbons (ethylene, ethane, propylene, propane and butane).

TABLE 3

| | | | | | | |
|---|---|---|---|---|---|---|
| Catalyst | Inlet | °C | 250 | 300 | 350 | 400 |
| temperature | Top | °C | 251 | 296 | 348 | 392 |
| | Middle | °C | 253 | 297 | 349 | 394 |
| | Bottom | °C | 257 | 301 | 352 | 398 |
| Gas concentration | H₂ | vol % | 6.1 | 11.1 | 12.2 | 14.3 |
| | CO | vol % | 2.4 | 5.8 | 2.9 | 2.1 |
| | CO₂ | vol % | 0.4 | 0.8 | 2.0 | 3.1 |
| | CH₄ | vol % | 2.2 | 6.0 | 3.9 | 3.2 |
| | N₂ | vol % | 86.5 | 73.9 | 75.1 | 69.2 |

| | | | | | |
|---|---|---|---|---|---|
| Ethanol conversion rate | % | 11.5 | 30.0 | 45.9 | 64.6 |

### Test Example 4

The test was conducted as in Test Example 1, except that the pressure in the reaction tube was 0.1 MPa (absolute pressure).

Table 4 shows, for each inlet temperature of the catalyst layer, temperatures of the top, middle, and bottom of the catalyst layer, the gas composition analysis after condensate separation, and the ethanol conversion rate.

The ethanol conversion rate was only 38.4% when the inlet temperature of the catalyst layer was 250 °C, but was 98.0% when the inlet temperature of the catalyst layer was 300 °C, and was 100% when the inlet temperature of the catalyst layer was 350 °C or more. The methane concentration of the produced gas was lower than that in Test Example 1. This is considered to be due to the fact that the reaction proceeds with an increase in temperature and the steam reforming reaction of ethanol proceeds rapidly when the temperature of the steam reforming catalyst layer is 400 °C or more.

TABLE 4

| | | | | | | |
|---|---|---|---|---|---|---|
| Catalyst | Inlet | °C | 250 | 300 | 350 | 400 |
| temperature | Top | °C | 250 | 381 | 412 | 447 |
| | Middle | °C | 256 | 412 | 436 | 466 |
| | Bottom | °C | 263 | 387 | 414 | 448 |
| Gas concentration | H₂ | vol % | 73.7 | 37.0 | 40.0 | 44.7 |
| | CO | vol % | 10.8 | 0.4 | 0.7 | 1.2 |
| | CO₂ | vol % | 0.1 | 8.3 | 9.2 | 10.2 |
| | CH₄ | vol % | 12.7 | 54.2 | 50.2 | 43.9 |

| | | | | | |
|---|---|---|---|---|---|
| Ethanol conversion rate | % | 38.4 | 98.0 | 100.0 | 100.0 |

### Evaluations of Test Examples 1 to 4

In Test Example 1, where hydrogen and water vapor were added to ethanol so that the molar ratio of hydrogen/ethanol was 2.0 and the molar ratio of water vapor/ethanol was 2.0, and the resultant was brought into contact with a catalyst containing ruthenium, ethanol was almost completely converted and a gas containing methane as the main component thereof was obtained if the inlet temperature of the catalyst layer was 250 °C or more. Also, when the resultant was brought into contact with the catalyst under conditions where the inlet temperature of the catalyst layer was 300 °C or more and the maximum temperature inside the catalyst layer was 370 °C or more, ethanol could be completely converted into a gas containing methane as the main component thereof.

In Test Example 2, where the molar ratio of hydrogen/ethanol was 1.0, the conversion rate of ethanol was substantially the same as in the case where the molar ratio of hydrogen/ethanol was 2.0, but a large amount of carbon dioxide remained in the produced gas.

Also, in Test Example 3, where no hydrogen was added, ethanol was not completely converted even when the inlet temperature of the catalyst layer was 400 °C (temperature inside the catalyst layer was between 392 °C and 398 °C). As disclosed in JP 2009-227588A, in a simple steam reforming reaction of ethanol without addition of hydrogen, a temperature of 400 °C or more is required to obtain sufficient ethanol conversion.

In Test Example 4, where the reaction pressure was 0.1 MPa, the ethanol conversion rate was lower in the temperature range of 300 °C or less, compared to Test example 1. It was confirmable that the reaction rate of the steam reforming reaction was reduced. One of the reasons why the methane concentration in the produced gas was lower than that in Test Example 1 may be that the equilibrium conversion rate of the methanation reaction is higher the higher the reaction pressure is.

From the above points, it is understood that the molar ratio of hydrogen/ethanol is preferably 1.0 or more and a reaction pressure is preferably higher than the ambient pressure in terms of obtaining a sufficient ethanol conversion ratio even if the steam reforming process is performed at a relatively low inlet temperature of the catalyst layer of about 250 °C to 300 °C.

Examples 1 and 2 are examples in which an elongated reaction tube was filled with two layers of catalysts on the inlet and outlet sides of the tube, a raw material gas containing ethanol, hydrogen, and water vapor was brought into contact with each catalyst layer at different temperatures, a steam reforming reaction was performed with the inlet side defined as the steam reforming reaction unit 1, and a methanation reaction was performed with the outlet side defined as the methanation reaction unit 2, thereby attempting to produce a high calorific value fuel gas.

### Example 1

A sheath tube (outer diameter 6 mm) for temperature measurement was passed through the center of a stainless steel reaction tube (inner diameter 20 mm), and 12 mL (about 9 g) of catalyst with 2 mass % ruthenium supported on active alumina supports (spherical shape of 2 to 4 mm) was filled between the reaction tube and the sheath tube to form a steam reforming catalyst layer. Above the steam reforming catalyst layer, alumina balls were filled to a height of 30 mm. Below the steam reforming catalyst layer, alumina balls were filled to a height of 145 mm to serve as a cooling zone for cooling the outlet gas of the steam reforming catalyst layer, and further below, 43 mL (about 33 g) of catalyst with 2 mass % ruthenium supported on active alumina supports (spherical shape of 2 to 4 mm) was filled to form a methanation catalyst layer. Thermocouples for temperature measurement were respectively installed in the sheath tube at the center of the alumina ball layer above the steam reforming catalyst layer, at the top, middle, and bottom of the steam reforming catalyst layer, and at the top, middle, and bottom of the methanation catalyst layer.

This reaction tube was loaded into an electric furnace. The electric furnace consists of three zones, namely, upper, middle, and lower stages, and the heating output of each stage can be controlled individually. The reaction tube was loaded into the electric furnace so that the lower edge of the steam reforming catalyst layer was located at the same height as the lower edge of the upper-stage heater of the electric furnace and the upper and lower edges of the lower-stage heater were located at the same height as the upper and lower edges of the methanation catalyst layer.

The upper-stage heater was controlled so that the temperature at the center of the alumina ball layer above the steam reforming catalyst layer was approximately 250 °C, and the lower-stage heater was controlled so that the temperature at the outlet of the methanation catalyst layer was 250 °C, and a reduction treatment was performed for 1 hour by passing a reducing gas that is a mixture of nitrogen gas and 2% (by volume) hydrogen gas.

After the above-described reduction treatment, while the pressure in the reaction tube was maintained at 0.7 MPa (absolute pressure), the upper-stage heater was controlled so that the temperature at the center of the alumina ball layer above the steam reforming catalyst layer (inlet temperature of the steam reforming catalyst layer) was 300 °C, and the lower-stage heater was controlled so that the outlet (bottom) of the methanation catalyst layer was 270 °C, a gas that is a mixture of ethanol at 200 mL/min, hydrogen at 400 mL/min, and water vapor at 400 mL/min was passed through the catalyst layer, from the top to the bottom. The outlet gas of the catalyst layer was analyzed in the same manner as in Test Example 1.

Table 5 shows, at 1, 2, 3, and 4 hours after the start of the reaction, the inlet temperatures of the steam reforming catalyst layer, the temperatures at the top, middle, and bottom of the steam reforming catalyst layer, the temperatures at the top, middle, and bottom of the methanation catalyst layer, the analysis results of the gas composition after condensate separation, and ethanol conversion rates.

One hour after the start of the reaction, the inlet temperature of the steam reforming catalyst layer was 295 °C, the temperature of the top of the steam reforming catalyst was 392 °C, and the outlet (bottom) temperature of the methanation catalyst layer was 269 °C. After condensate separation, the gas contained 94.2% methane, 5.3% hydrogen, and 0.5% carbon dioxide, and thus was a methane-dominant gas that contains methane as the main component thereof, and small amounts of hydrogen and carbon dioxide. The ethanol conversion rate was 100%.

After 2 hours from the start of the reaction, the inlet temperature of the steam reforming catalyst layer was controlled stably at 299 °C to 300 °C, the temperature of the top of the steam reforming catalyst layer was 397 °C to 401 °C, and the outlet (bottom) temperature of the methanation catalyst layer was 270 °C to 271 °C, so the reaction continued stably. After condensate separation, the gas remained stable at 95% methane, 4% hydrogen, and 1% carbon dioxide, resulting in a gas with high methane purity that can be easily used as a raw material for city gas. The ethanol conversion rate remained at 100%.

TABLE 5

| | | | Elapsed time | | | |
|---|---|---|---|---|---|---|
| | | | 1 h | 2 h | 3 h | 4 h |
| Steam reforming | Inlet | °C | 295 | 299 | 300 | 299 |
| catalyst | Top | °C | 392 | 397 | 400 | 401 |
| temperature | Middle | °C | 363 | 370 | 374 | 374 |
| | Bottom | °C | 333 | 341 | 345 | 346 |
| Methanation | Top | °C | 263 | 271 | 274 | 275 |
| catalyst | Middle | °C | 277 | 281 | 282 | 283 |
| temperature | Bottom | °C | 269 | 270 | 270 | 271 |
| Gas concentration | H₂ | vol % | 5.3 | 4.2 | 4.2 | 4.2 |
| | CO | vol % | 0 | 0 | 0 | 0 |
| | CO₂ | vol % | 0.5 | 0.8 | 1.0 | 0.9 |
| | CH₄ | vol % | 94.2 | 95.0 | 94.8 | 94.9 |

| | | | | | |
|---|---|---|---|---|---|
| Ethanol conversion rate | % | 100.0 | 100.0 | 100.0 | 100.0 |

### Example 2

The test was conducted as in Test Example 1, except that a gas obtained by mixing ethanol at 200 mL/min, hydrogen at 200 mL/min, and water vapor at 400 mL/min was used as the gas supplied to the steam reforming catalyst layer.

Table 6 shows, at 1, 2, 3, and 4 hours after the start of the reaction, the inlet temperatures of the steam reforming catalyst layer, the temperatures at the top, middle, and bottom of the steam reforming catalyst layer, the temperatures at the top, middle, and bottom of the methanation catalyst layer, the analysis results of the gas composition after condensate separation, and ethanol conversion rates.

The ethanol conversion rate remained stable at 100%. After condensate separation, the composition of the gas remained stable at 85.5% methane, 1.9% hydrogen, and 12.6% carbon dioxide. The resulting gas contains methane as the main component thereof, but contains a slightly higher concentration of carbon dioxide. It is difficult for the gas to be directly used as a raw material for city gas in a typical city gas pipeline network, but the gas may be used as a raw material for city gas in a city gas pipeline network where the upper limit of allowable carbon dioxide concentration is high.

TABLE 6

| | | | Elapsed time | | | |
|---|---|---|---|---|---|---|
| | | | 1 h | 2 h | 3 h | 4 h |
| Steam reforming | Inlet | °C | 299 | 299 | 299 | 301 |
| catalyst | Top | °C | 383 | 387 | 388 | 389 |
| temperature | Middle | °C | 354 | 360 | 363 | 363 |
| | Bottom | °C | 322 | 330 | 333 | 334 |
| Methanation | Top | °C | 264 | 270 | 272 | 273 |
| catalyst | Middle | °C | 280 | 282 | 284 | 283 |
| temperature | Bottom | °C | 270 | 271 | 271 | 270 |
| Gas concentration | H₂ | vol % | 1.9 | 1.9 | 1.9 | 1.9 |
| | CO | vol % | 0 | 0 | 0 | 0 |
| | CO₂ | vol % | 12.6 | 12.6 | 12.6 | 12.6 |
| | CH₄ | vol % | 85.5 | 85.5 | 85.5 | 85.5 |

| | | | | | |
|---|---|---|---|---|---|
| Ethanol conversion rate | % | 100.0 | 100.0 | 100.0 | 100.0 |

The following will describe examples based on process calculations. Pressure loss and heat dissipation loss in each device and conduit are not considered.

### Example 3

In this example, as shown in FIG. 3, the raw material gas conditioning unit 3 is provided to condition the composition of a raw material gas before the raw material gas is supplied to the steam reforming reaction unit 1. To the raw material gas conditioning unit 3, ethanol, hydrogen, and water are supplied at a molar ratio of 1:2:1.2 and a pressure of 0.7 MPa (absolute pressure). In the raw material gas conditioning unit 3, these components are mixed and heated to 300 °C, and the conditioned gas obtained by conditioning the raw material gas is fed to the steam reforming reaction unit 1.

In the steam reforming reaction unit 1, the raw material gas comes into contact with the steam reforming catalyst under an adiabatic condition, a steam reforming reaction of ethanol, a CO shift reaction, and a methanation reaction proceed to reach a chemical equilibrium state, and the produced gas is cooled to 250 °C and is fed into the methanation reaction unit 2.

In the methanation reaction unit 2, the CO shift reaction and the methanation reaction proceed under an isothermal condition of 250 °C, reaching a chemical equilibrium state and yielding a fuel gas that contains methane and water vapor as the main components thereof. When this fuel gas is cooled and water is condensed and separated, a fuel gas is obtained that contains methane as the main component thereof, and small amounts of hydrogen and carbon dioxide.

For each reaction unit, the inlet and outlet temperatures, as well as the composition (by volume after desiccation) and the carbon activity of the outlet gas are shown in Table 7. Here, the carbon activity is calculated as K_{P}/(P_{CO2}/P_{CO}²) using the partial pressures (P_{CO} and P_{CO2}) of carbon monoxide and carbon dioxide of the outlet gas and the equilibrium constant (K_{P}) of the thermodynamically calculated disproportionation reaction of carbon monoxide (Formula 7), and if this value is greater than 1, a carbon deposition reaction progresses in view of chemical equilibrium.

2CO->C(solid)+CO₂ (Formula 7)

TABLE 7

| | | | Steam | |
|---|---|---|---|---|
| | | | reforming | Methanation |
| Inlet temperature (°C) | | | 300 | 250 |
| Outlet temperature | | | 619 | 250 |
| (°C) | | | | |
| | Outlet | CH₄ | 39.44 | 96.20 |
| | composition | H₂ | 47.48 | 3.04 |
| | (%) | CO | 4.85 | 0.00 |
| | | CO₂ | 8.23 | 0.76 |

| | | |
|---|---|---|
| Carbon activity | 0.981 | 0.276 |

The outlet temperature of the steam reforming reaction unit 1 is 619 °C. The gas composition at the outlet of the steam reforming reaction unit 1 contains 39.44% methane and 47.48% hydrogen. Thus, the methane concentration is less than 40%, meaning that the gas contains a high concentration of hydrogen.

The gas composition at the outlet of the methanation reaction unit 2 contains 96.20% methane, 3.04% hydrogen, and 0.76% carbon dioxide, meaning that the gas is a high methane purity fuel gas (high calorific value fuel gas) sufficient to be used as a raw material for city gas.

Both at the outlet of the steam reforming reaction unit 1 and at the outlet of the methanation reaction unit 2, the carbon activity is below 1, and carbon deposition is avoided in chemical equilibrium.

Note that in the description above, it is assumed that the steam reforming reaction reacts under an adiabatic condition and the methanation reaction reacts under an isothermal condition, but even in reactions in adiabatic reactors, a certain amount of heat dissipation from the reactor surface may occur, and a temperature rise may also occur locally in heat exchanger reactors. The temperature of the catalyst layer of the methanation catalyst layer is 250 °C or more, and the peak temperature may be higher than 250 °C by about 100 °C to 200 °C, depending on the balance between heat generation by the reaction and heat removal by heat exchange. The outlet temperature of the steam reforming catalyst layer is 619 °C if the thermal insulation is perfect, but may be lower than 619 °C by 100 °C to 200 °C if the thermal insulation is not perfect.

Therefore, the minimum temperature of the catalyst layer is not lower than 250 °C and the maximum temperature is not higher than 619 °C through both the steam reforming catalyst and the methanation catalyst. In this regard, it is desirable that the carbon activity be below 1 at a molar ratio of ethanol, hydrogen, and water of 1:2:1.2 and a pressure of 0.7 MPa, under conditions where the steam reforming reaction of ethanol, the CO shift reaction, and the methanation reaction reached equilibrium in the entire temperature range between 250 °C and 619 °C, inclusive.

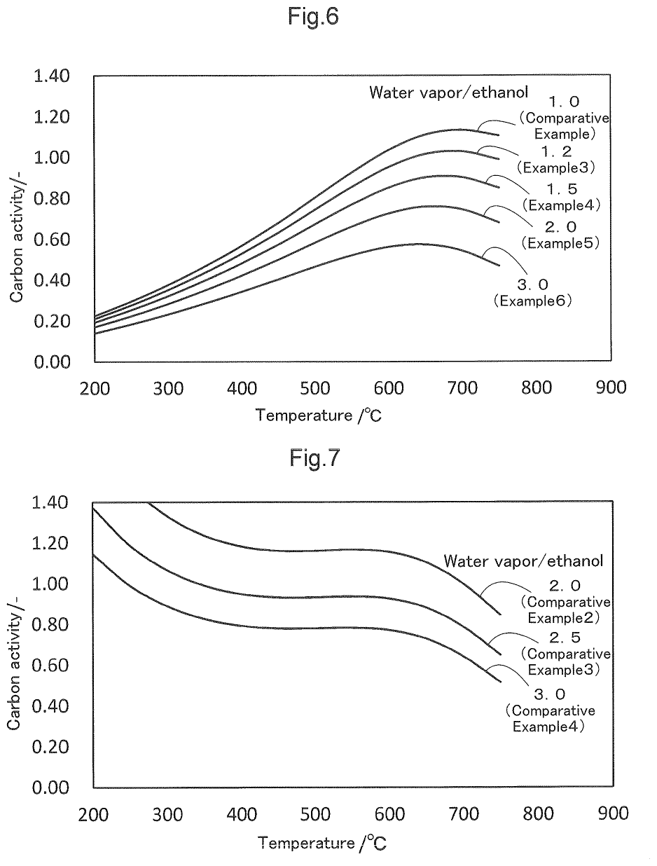

FIG. 6 (water vapor/ethanol=1.2) shows the carbon activities when the chemical equilibrium was reached at the temperatures. Since the carbon activity is below 1 in the entire temperature range of 250 °C to 619 °C, carbon deposition is avoided in chemical equilibrium under adiabatic, isothermal, and intermediate conditions between the two conditions.

### Example 4

Calculation was performed as in Example 3, except that the molar ratio of ethanol, hydrogen, and water supplied to the raw material gas conditioning unit 3 was defined as ethanol:hydrogen:water=1:2:1.5.

For each reaction unit, the inlet and outlet temperatures, as well as the composition (by volume after desiccation) and the carbon activity of the outlet gas are shown in Table 8.

TABLE 8

| | | | Steam | |
|---|---|---|---|---|
| | | | reforming | Methanation |
| Inlet temperature (°C) | | | 300 | 250 |
| Outlet temperature | | | 608 | 250 |
| (°C) | | | | |
| | Outlet | CH₄ | 39.14 | 95.90 |
| | composition | H₂ | 47.86 | 3.28 |
| | (%) | CO | 4.15 | 0.00 |
| | | CO₂ | 8.85 | 0.82 |

| | | |
|---|---|---|
| Carbon activity | 0.861 | 0.253 |

The outlet temperature of the steam reforming reaction unit 1 is 608 °C. The gas composition at the outlet of the steam reforming reaction unit 1 contains 39.14% methane and 47.86% hydrogen. Due to an increase in the amount of water vapor added, it is difficult for a methanation reaction with water serving as the product to proceed in equilibrium, and thus the concentration of methane is slightly lower than in Example 1.

The gas composition at the outlet of the methanation reaction unit 2 contains 95.90% methane, 3.28% hydrogen, and 0.82% carbon dioxide, meaning that the concentration of methane is slightly reduced but a high methane purity fuel gas (high calorific value fuel gas) sufficient to be used as a raw material for city gas is obtained.

Both at the outlet of the steam reforming reaction unit 1 and at the outlet of the methanation reaction unit 2, the carbon activity is below 1, and is lower than in Example 3. The reason is considered that carbon deposition is less likely to occur as the amount of water vapor added increases. As shown in FIG. 6 (water vapor/ethanol=1.5), the carbon activity is below 1 in the entire temperature range between 250 °C and 608 °C, and carbon deposition does not occur in view of equilibrium over the entire temperature range assumed for the steam reforming and methanation reactions.

### Example 5

Calculation was performed as in Example 3, except that the molar ratio of ethanol, hydrogen, and water supplied to the raw material gas conditioning unit 3 was defined as ethanol:hydrogen:water=1:2:2.

For each reaction unit, the inlet and outlet temperatures, as well as the composition (by volume after desiccation) and the carbon activity of the outlet gas are shown in Table 9.

TABLE 9

| | | | Steam | |
|---|---|---|---|---|
| | | | reforming | Methanation |
| Inlet temperature (°C) | | | 300 | 250 |
| Outlet temperature | | | 591 | 250 |
| (°C) | | | | |
| | Outlet | CH₄ | 38.83 | 95.42 |
| | composition | H₂ | 48.28 | 3.66 |
| | (%) | CO | 3.25 | 0.00 |
| | | CO₂ | 9.64 | 0.92 |

| | | |
|---|---|---|
| Carbon activity | 0.714 | 0.223 |

The gas composition at the outlet of the methanation reaction unit 2 contains 95.42% methane, 3.66% hydrogen, and 0.92% carbon dioxide, meaning that the concentration of methane is slightly reduced compared to Example 4, but a high methane purity fuel gas (high calorific value fuel gas) sufficient to be used as a raw material for city gas is obtained.

It can be seen that this gas composition is almost identical to the gas composition (94.9% methane, 4.2% hydrogen, and 0.9% carbon dioxide) obtained in Example 1, which has the same molar ratios of ethanol, hydrogen, and water and substantially the same outlet temperature and reaction pressure of the methanation catalyst layer.

Both at the outlet of the steam reforming reaction unit 1 and at the outlet of the methanation reaction unit 2, the carbon activity is sufficiently lower than 1. As shown in FIG. 6 (water vapor/ethanol=2), the carbon activity is below 1 in the entire temperature range between 250 °C and 591 °C, and carbon deposition does not occur in view of equilibrium over the entire temperature range assumed for the steam reforming and methanation reactions.

### Example 6

Calculation was performed as in Example 3, except that the molar ratio of ethanol, hydrogen, and water supplied to the raw material gas conditioning unit 3 was defined as ethanol:hydrogen:water=1:2:3.

For each reaction unit, the inlet and outlet temperatures, as well as the composition (by volume after desiccation) and the carbon activity of the outlet gas are shown in Table 10.

TABLE 10

| | | | Steam | |
|---|---|---|---|---|
| | | | reforming | Methanation |
| Inlet temperature (°C) | | | 300 | 250 |
| Outlet temperature | | | 562 | 250 |
| (°C) | | | | |
| | Outlet | CH₄ | 38.66 | 94.51 |
| | composition | H₂ | 48.66 | 4.39 |
| | (%) | CO | 2.09 | 0.00 |
| | | CO₂ | 10.60 | 1.10 |

| | | |
|---|---|---|
| Carbon activity | 0.531 | 0.183 |

The outlet temperature of the steam reforming reaction unit 1 is 562 °C. Compared to Example 3, the outlet temperature is reduced by 50 °C or more, but this is because an increase in the added amount of water vapor mitigates the temperature increase, which is advantageous in terms of the durability of the steam reforming catalyst.

The gas composition at the outlet of the methanation reaction unit 2 contains 94.51% methane, 4.39% hydrogen, and 1.10% carbon dioxide, meaning that the concentration of methane is slightly reduced compared to Example 5, but a high methane purity fuel gas (high calorific value fuel gas) sufficient to be used as a raw material for city gas is obtained.

Both at the outlet of the steam reforming reaction unit 1 and at the outlet of the methanation reaction unit 2, the carbon activity is sufficiently lower than 1. Also, as shown in FIG. 6 (water vapor/ethanol=3), the carbon activity is below 1 in the entire temperature range between 250 °C and 562 °C, and carbon deposition does not occur in view of equilibrium over the entire temperature range assumed for the steam reforming and methanation reactions.

### Comparative Example 1

Calculation was performed as in Example 3, except that the molar ratio of ethanol, hydrogen, and water supplied to the raw material gas conditioning unit 3 was defined as ethanol:hydrogen:water=1:2:1.

For each reaction unit, the inlet and outlet temperatures, as well as the composition (by volume after desiccation) and the carbon activity of the outlet gas are shown in Table 11.

TABLE 11

| | | | Steam | |
|---|---|---|---|---|
| | | | reforming | Methanation |
| Inlet temperature (°C) | | | 300 | 250 |
| Outlet temperature | | | 628 | 250 |
| (°C) | | | | |
| | Outlet | CH₄ | 39.71 | 96.41 |
| | composition | H₂ | 47.15 | 2.87 |
| | (%) | CO | 5.40 | 0.00 |
| | | CO₂ | 7.74 | 0.72 |

| | | |
|---|---|---|
| Carbon activity | 1.081 | 0.295 |

The outlet temperature of the steam reforming reaction unit 1 is 628 °C. Also, at the outlet of the steam reforming reaction unit 1, the carbon activity exceeded 1. Compared to the Examples, due to the lower amount of water vapor added, the outlet temperature of the steam reforming reaction unit 1 increased and the carbon activity level exceeded 1, causing the risk of degradation of the steam reforming catalyst due to thermal degradation and carbon deposition.

As shown in FIG. 6 (water vapor/ethanol=1), at 580 °C or less, carbon deposition does not occur in equilibrium, and therefore, it may be possible to avoid carbon deposition by proceeding with the reaction while removing heat, but locally, the temperature may reach a temperature close to the exit temperature of the adiabatic reaction, which presents certain difficulties in controlling the reaction.

### Comparative Example 2

Calculation was performed as in Example 3, except that the molar ratio of ethanol and water supplied to the raw material gas conditioning unit 3 was defined as ethanol:water=1:2 without addition of hydrogen.

For each reaction unit, the inlet and outlet temperatures, as well as the composition (by volume after desiccation) and the carbon activity of the outlet gas are shown in Table 12.

TABLE 12

| | | | Steam | |
|---|---|---|---|---|
| | | | reforming | Methanation |
| Inlet temperature (°C) | | | 300 | 250 |
| Outlet temperature | | | 518 | 250 |
| (°C) | | | | |
| | Outlet | CH₄ | 49.36 | 73.87 |
| | composition | H₂ | 25.09 | 1.13 |
| | (%) | CO | 2.21 | 0.00 |
| | | CO₂ | 23.34 | 25.00 |

| | | |
|---|---|---|
| Carbon activity | 1.162 | 1.485 |

The outlet temperature of the steam reforming reaction unit 1 is 518 °C. The gas composition at the outlet of the steam reforming reaction unit 1 contains 49.36% methane and 25.09% hydrogen. The methane concentration is higher than in Example 1. This may be due to the fact that the outlet temperature is lower than in Example 1, which facilitated the methanation reaction in equilibrium.

On the other hand, the gas composition at the outlet of the methanation reaction unit 2 contains 73.87% methane, 1.13% hydrogen, and 25.00% carbon dioxide, meaning that it is difficult to use the gas as a raw material for city gas unless carbon dioxide is removed.

A further problem is that the carbon activity exceeded 1 at both the outlet of the steam reforming reaction unit 1 and the outlet of the methanation reaction unit 2, which raises concerns about degradation of the steam reforming catalyst and the methanation catalyst due to carbon deposition. FIG. 7 (water vapor/ethanol=2.0) shows the carbon activities when the chemical equilibrium was reached at the corresponding temperatures. The carbon activity exceeds 1 in the entire temperature range of 700 °C or less, which means that carbon deposition proceeds in view of chemical equilibrium under adiabatic, isothermal, and intermediate conditions between the two conditions.

### Comparative Example 3

Calculation was performed as in Example 2, except that the molar ratio of ethanol and water supplied to the raw material gas conditioning unit 3 was defined as ethanol:water=1:2.5.

For each reaction unit, the inlet and outlet temperatures, as well as the composition (by volume after desiccation) and the carbon activity of the outlet gas are shown in Table 13.

TABLE 13

| | | | Steam | |
|---|---|---|---|---|
| | | | reforming | Methanation |
| Inlet temperature (°C) | | | 300 | 250 |
| Outlet temperature | | | 502 | 250 |
| (°C) | | | | |
| | Outlet | CH₄ | 49.02 | 73.66 |
| | composition | H₂ | 25.58 | 1.33 |
| | (%) | CO | 1.60 | 0.00 |
| | | CO₂ | 23.80 | 25.00 |

| | | |
|---|---|---|
| Carbon activity | 0.932 | 1.189 |

The outlet temperature of the steam reforming reaction unit 1 is 502 °C, and the carbon activity is lower than 1.

The gas composition at the outlet of the methanation reaction unit 2 contains 73.66% methane, 1.33% hydrogen, and 25.00% carbon dioxide, meaning that it is impossible to use the gas as a raw material for city gas unless carbon dioxide is removed. The carbon activity exceeded 1 at the outlet of the methanation reaction unit 2, which raises concerns about degradation of the methanation catalyst due to carbon deposition. FIG. 7 (water vapor/ethanol=2.5) shows the carbon activities when the chemical equilibrium was reached at the corresponding temperatures. At 340 °C or less, the carbon activity exceeds 1, and thus if the methanation reaction is conducted at a low temperature to allow the methanation to proceed, carbon deposition will proceed in view of chemical equilibrium.

### Comparative Example 4

Calculation was performed as in Example 2, except that the molar ratio of ethanol and water supplied to the raw material gas conditioning unit 3 was defined as ethanol:water=1:3.

For each reaction unit, the inlet and outlet temperatures, as well as the composition (by volume after desiccation) and the carbon activity of the outlet gas are shown in Table 14.

TABLE 14

| | | | Steam | |
|---|---|---|---|---|
| | | | reforming | Methanation |
| Inlet temperature (°C) | | | 300 | 250 |
| Outlet temperature | | | 488 | 250 |
| (°C) | | | | |
| | Outlet | CH₄ | 48.84 | 73.46 |
| | composition | H₂ | 25.86 | 1.54 |
| | (%) | CO | 1.21 | 0.00 |
| | | CO₂ | 24.10 | 25.00 |

| | | |
|---|---|---|
| Carbon activity | 0.779 | 0.991 |

The outlet temperature of the steam reforming reaction unit 1 is 488 °C.

The gas composition at the outlet of the methanation reaction unit 2 contains 73.46% methane, 1.54% hydrogen, and 25.00% carbon dioxide, meaning that it is difficult to use the gas as a raw material for city gas unless carbon dioxide is removed. Both at the outlet of the steam reforming reaction unit 1 and at the outlet of the methanation reaction unit 2, the carbon activity is below 1, and carbon deposition would be avoided in chemical equilibrium. The results of Comparative Examples 2 to 4 indicate that in simple steam reforming of ethanol without hydrogen, water vapor needs to be added so that the molar ratio of water vapor to ethanol is 3 or more (or the molar ratio of water vapor to the methane to be produced is 2 or more) to avoid carbon deposition in equilibrium. Note that as shown in FIG. 5 (water vapor/ethanol=3.0), at 245 °C or less, the carbon activity exceeds 1, and thus if the methanation reaction is conducted at a temperature not higher than 245 °C to allow the methanation to proceed, carbon deposition may proceed in view of chemical equilibrium.

### Example 7

Calculation was performed as in Example 3, except that the molar ratio of ethanol, hydrogen, and water supplied to the raw material gas conditioning unit 3 was defined as ethanol:hydrogen:water=1:2.5:2.

For each reaction unit, the inlet and outlet temperatures, as well as the composition (by volume after desiccation) and the carbon activity of the outlet gas are shown in Table 15.

TABLE 15

| | | | Steam | |
|---|---|---|---|---|
| | | | reforming | Methanation |
| Inlet temperature (°C) | | | 300 | 250 |
| Outlet temperature | | | 601 | 250 |
| (°C) | | | | |
| | Outlet | CH₄ | 36.76 | 80.00 |
| | composition | H₂ | 52.35 | 20.00 |
| | (%) | CO | 3.13 | 0.00 |
| | | CO₂ | 7.76 | 0.00 |

| | | |
|---|---|---|
| Carbon activity | 0.644 | 0.006 |

The gas composition at the outlet of the steam reforming reaction unit 2 contained 80.00% methane and 20.00% hydrogen, which means that the gas contains a relatively high concentration of hydrogen but the concentrations of carbon monoxide and carbon dioxide were less than 0.01%.

Both at the outlet of the steam reforming reaction unit 1 and at the outlet of the methanation reaction unit 2, the carbon activity is sufficiently lower than 1.

### Example 8

Calculation was performed as in Example 3, except that the molar ratio of ethanol, hydrogen, and water supplied to the raw material gas conditioning unit 3 was defined as ethanol:hydrogen:water=1:1.5:2.

For each reaction unit, the inlet and outlet temperatures, as well as the composition (by volume after desiccation) and the carbon activity of the outlet gas are shown in Table 16.

TABLE 16

| | | | Steam | |
|---|---|---|---|---|
| | | | reforming | Methanation |
| Inlet temperature (°C) | | | 300 | 250 |
| Outlet temperature | | | 579 | 250 |
| (°C) | | | | |
| | Outlet | CH₄ | 41.37 | 91.28 |
| | composition | H₂ | 43.43 | 2.08 |
| | (%) | CO | 3.22 | 0.00 |
| | | CO₂ | 11.98 | 6.64 |

| | | |
|---|---|---|
| Carbon activity | 0.792 | 0.626 |

The gas composition at the outlet of the steam reforming reaction unit 2 contained 91.28% methane, 2.08% hydrogen, and 6.64% carbon dioxide.

Both at the outlet of the steam reforming reaction unit 1 and at the outlet of the methanation reaction unit 2, the carbon activity is sufficiently lower than 1.

### Example 9

Calculation was performed as in Example 3, except that the molar ratio of ethanol, hydrogen, and water supplied to the raw material gas conditioning unit 3 was defined as ethanol:hydrogen:water=1:1:2.

For each reaction unit, the inlet and outlet temperatures, as well as the composition (by volume after desiccation) and the carbon activity of the outlet gas are shown in Table 17.

TABLE 17

| | | | Steam | |
|---|---|---|---|---|
| | | | reforming | Methanation |
| Inlet temperature (°C) | | | 300 | 250 |
| Outlet temperature | | | 563 | 250 |
| (°C) | | | | |
| | Outlet | CH₄ | 43.97 | 85.67 |
| | composition | H₂ | 38.01 | 1.63 |
| | (%) | CO | 3.07 | 0.00 |
| | | CO₂ | 14.95 | 12.70 |

| | | |
|---|---|---|
| Carbon activity | 0.889 | 0.918 |

The gas composition at the outlet of the steam reforming reaction unit 2 contained 85.67% methane, 1.63% hydrogen, and 12.70% carbon dioxide.

It can be seen that this gas composition is almost identical to the gas composition (85.5% methane, 1.9% hydrogen, and 12.6% carbon dioxide) obtained in Example 2, which has the same molar ratio of ethanol, hydrogen, and water and substantially the same outlet temperature and reaction pressure of the methanation catalyst layer.

Both at the outlet of the steam reforming reaction unit 1 and at the outlet of the methanation reaction unit 2, the carbon activity is lower than 1. Although, in Example 2, a methane-dominant gas was obtained stably without any change in gas composition over 4 hours, it is assumed that the stable reaction was enabled due to the fact that the carbon activity was sufficiently lower than 1 under the present reaction conditions.

### Example 10

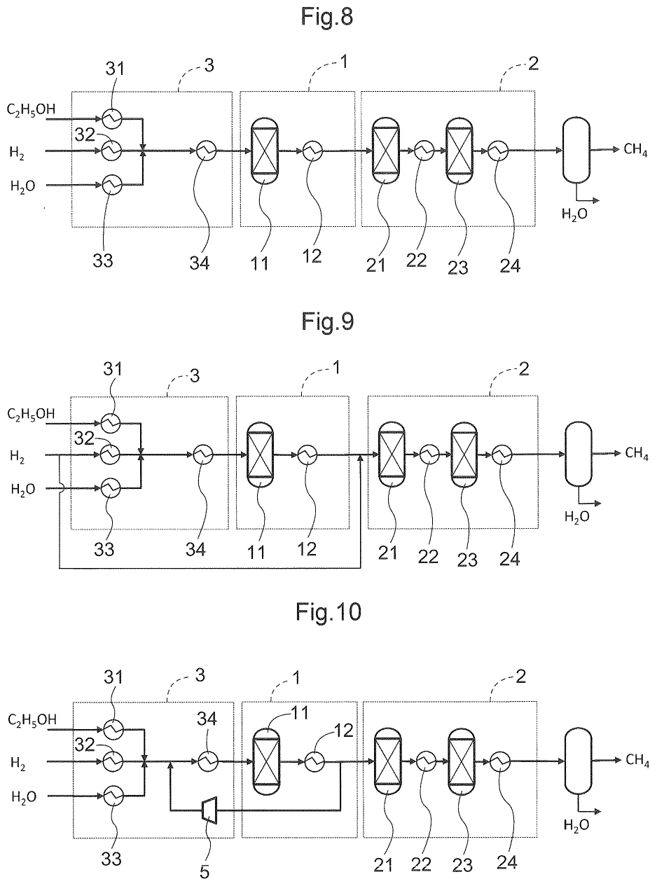

The present example shows a calculation result of a fuel gas production process. The process flow is shown in FIG. 8.

In the raw material gas conditioning unit 3, where the raw material gas conditioning process is performed, ethanol, hydrogen, and water are respectively supplied at flow rates of 1 mol/s, 2.0 mol/s, and 2.0 mol/s, at a temperature of 25 °C and a pressure of 0.8 MPa. The raw gas conditioning unit 3 includes heat exchangers 31 to 34.

Ethanol, hydrogen, and water are heated and mixed to form a raw material gas, which is further heated to 300 °C and fed into the steam reforming reaction unit 1.

The steam reforming reaction unit 1, where the steam reforming process is performed, includes a steam reforming reactor 11 and a heat exchanger 12. The raw material gas is fed into the steam reforming reactor 11, where a steam reforming reaction of ethanol, a CO shift reaction, and a methanation reaction proceed adiabatically. The outlet gas from the steam reforming reactor 11 is cooled to 250 °C in the heat exchanger 12 and then fed to the methanation reaction unit 2.

The methanation reaction unit 2, where the methanation process is performed, includes a first methanation reactor 21, a heat exchanger 22, a second methanation reactor 23, and a heat exchanger 24.

The first methanation reactor 21 is an adiabatic reactor, and the outlet gas thereof is cooled to 250 °C in the heat exchanger 22 and then fed to the second methanation reactor 23. The second methanation reactor 23 is a heat exchanger reactor, where a methanation reaction proceeds at nearly 250 °C to an equilibrium composition of 250 °C. The outlet gas from the second methanation reactor 23 is cooled in the heat exchanger 24, and after separation of water and desiccation, a fuel gas that contains methane as the main component thereof is obtained.

Temperatures and flow rates at key positions in the process are shown in Table 18.

The composition of the obtained fuel gas (by volume after desiccation) contains 95.65% methane, 3.48% hydrogen, 0.87% carbon dioxide, and 2 ppm carbon monoxide. It is apparent that a fuel gas with high methane purity (high calorific value fuel gas) that can be used as city gas can be obtained by the method of the present invention.

Also, since the raw material gas is heated to 300 °C and fed into the steam reforming reaction unit 1, and the methanation reaction proceeds in the second methanation reactor 23 at nearly 250 °C, the carbon activity is lower than 1 at the outlet of each reactor, and carbon deposition is expected to be avoided in chemical equilibrium.

TABLE 18

| | Temperature | Pressure | Flow rate [mol/s] | | | | | |
|---|---|---|---|---|---|---|---|---|
| | °C | MPa | H₂ | CO | CO₂ | H₂O | CH₄ | C₂H₅OH |
| Inlet of raw | 25 | 0.8 | 2.000 | 0.000 | 0.000 | 2.000 | 0.000 | 1.000 |
| material gas | | | | | | | | |
| conditioning | | | | | | | | |
| unit | | | | | | | | |
| Inlet of | 300 | 0.8 | 2.000 | 0.000 | 0.000 | 2.000 | 0.000 | 1.000 |
| steam | | | | | | | | |
| reforming | | | | | | | | |
| reactor | | | | | | | | |
| Outlet of | 596 | 0.8 | 1.833 | 0.124 | 0.365 | 2.146 | 1.511 | 0.000 |
| steam | | | | | | | | |
| reforming | | | | | | | | |
| reactor | | | | | | | | |
| Inlet of first | 250 | 0.8 | 1.833 | 0.124 | 0.365 | 2.146 | 1.511 | 0.000 |
| methanation | | | | | | | | |
| reactor | | | | | | | | |
| Outlet of | 479 | 0.8 | 0.855 | 0.012 | 0.205 | 2.579 | 1.783 | 0.000 |
| first | | | | | | | | |
| methanation | | | | | | | | |
| reactor | | | | | | | | |
| Inlet of | 250 | 0.8 | 0.855 | 0.012 | 0.205 | 2.579 | 1.783 | 0.000 |
| second | | | | | | | | |
| methanation | | | | | | | | |
| reactor | | | | | | | | |
| Outlet of | 250 | 0.8 | 0.072 | 0.000 | 0.018 | 2.964 | 1.982 | 0.000 |
| second | | | | | | | | |
| methanation | | | | | | | | |
| reactor | | | | | | | | |
| Fuel gas | 25 | 0.8 | 0.072 | 0.000 | 0.018 | 0.000 | 1.982 | 0.000 |
| (after | | | | | | | | |
| desiccation) | | | | | | | | |

### Example 11

Calculation was performed as in Example 10, except that the flow rate of hydrogen was changed from 2.0 mol/s to 1.0 mol/s.

Temperatures and flow rates at key positions in the process are shown in Table 19.

The composition of the obtained fuel gas (by volume after desiccation) contains 85.78% methane, 1.53% hydrogen, 12.69% carbon dioxide, and 18 ppm carbon monoxide.

Also, since the raw material gas is heated to 300 °C and fed into the steam reforming reaction unit 1, and the methanation reaction proceeds in the second methanation reactor 23 at nearly 250 °C, the carbon activity is lower than 1 at the outlet of each reactor, and carbon deposition is expected to be avoided in chemical equilibrium.

TABLE 19

| | Temperature | Pressure | Flow rate [mol/s] | | | | | |
|---|---|---|---|---|---|---|---|---|
| | °C | MPa | H₂ | CO | CO₂ | H₂O | CH₄ | C₂H₅OH |
| Inlet of raw | 25 | 0.8 | 1.000 | 0.000 | 0.000 | 2.000 | 0.000 | 1.000 |
| material gas | | | | | | | | |
| conditioning | | | | | | | | |
| unit | | | | | | | | |
| Inlet of steam | 300 | 0.8 | 1.000 | 0.000 | 0.000 | 2.000 | 0.000 | 1.000 |
| reforming | | | | | | | | |
| reactor | | | | | | | | |
| Outlet of | 568 | 0.8 | 1.204 | 0.099 | 0.477 | 1.947 | 1.424 | 0.000 |
| steam | | | | | | | | |
| reforming | | | | | | | | |
| reactor | | | | | | | | |
| Inlet of first | 250 | 0.8 | 1.204 | 0.099 | 0.477 | 1.947 | 1.424 | 0.000 |
| methanation | | | | | | | | |
| reactor | | | | | | | | |
| Outlet of first | 448 | 0.8 | 0.463 | 0.010 | 0.358 | 2.274 | 1.632 | 0.000 |
| methanation | | | | | | | | |
| reactor | | | | | | | | |
| Inlet of second | 250 | 0.8 | 0.463 | 0.010 | 0.358 | 2.274 | 1.632 | 0.000 |
| methanation | | | | | | | | |
| reactor | | | | | | | | |
| Outlet of | 250 | 0.8 | 0.031 | 0.000 | 0.258 | 2.484 | 1.742 | 0.000 |
| second | | | | | | | | |
| methanation | | | | | | | | |
| reactor | | | | | | | | |
| Fuel gas | 25 | 0.8 | 0.031 | 0.000 | 0.258 | 0.000 | 1.742 | 0.000 |
| (after | | | | | | | | |
| desiccation) | | | | | | | | |

### Example 12

The present example shows a calculation result of a fuel gas production process in the form shown in the block flow diagram of FIG. 4. The process flow is shown in FIG. 9.

In the raw material gas conditioning unit 3, where the raw material gas conditioning process is performed, ethanol, hydrogen, and water are respectively supplied at flow rates of 1 mol/s, 1.0 mol/s, and 2.0 mol/s, at a temperature of 25 °C and a pressure of 0.8 MPa. The raw gas conditioning unit 3 includes the heat exchangers 31 to 34.

Ethanol, hydrogen, and water are heated and mixed to form a raw material gas, which is further heated to 300 °C and fed into the steam reforming reaction unit 1.

The steam reforming reaction unit 1, where the steam reforming process is performed, includes the steam reforming reactor 11 and the heat exchanger 12. The raw material gas is fed into the steam reforming reactor 11, where a steam reforming reaction of ethanol, a CO shift reaction, and a methanation reaction proceed adiabatically. The outlet gas from the steam reforming reactor 11 is cooled to a predetermined temperature in the heat exchanger 12 and hydrogen is added at 1.0 mol/s from a hydrogen supply line leading to the raw material conditioning unit 3. Then, the resulting gas is fed to the methanation reaction unit 2 at 250 °C.

The methanation reaction unit 2, where the methanation process is performed, includes the first methanation reactor 21, the heat exchanger 22, the second methanation reactor 23, and the heat exchanger 24.

The first methanation reactor 21 is an adiabatic reactor, and the outlet gas thereof is cooled to 250 °C in the heat exchanger 22 and then fed to the second methanation reactor 23. The second methanation reactor 23 is a heat exchanger reactor, where a methanation reaction proceeds at nearly 250 °C to an equilibrium composition of 250 °C. The outlet gas from the second methanation reactor 23 is cooled in the heat exchanger 24, and after separation of water and desiccation, a fuel gas that contains methane as the main component thereof is obtained.

Temperatures and flow rates at main positions in the process are shown in Table 20.

The composition of the obtained fuel gas (by volume after desiccation) contains 95.65% methane, 3.48% hydrogen, 0.87% carbon dioxide, and 2 ppm carbon monoxide. It is apparent that a fuel gas with high methane purity (high calorific value fuel gas) that can be used as city gas can be obtained by the method of the present invention. At the outlet of each reactor, the carbon activity is lower than 1, and carbon deposition is avoided in chemical equilibrium.

Comparing Example 10 and Example 12, the resulting fuel gas has the same composition. On the other hand, the temperature at the outlet of the steam reforming reactor is 596 °C in Example 10, and is 568 °C in Example 12, which is about 30 °C lower. This is because under the conditions of Example 12, hydrogen is stoichiometrically insufficient to methane all the carbons in the ethanol, and the exothermic methanation reaction proceeds slowly. Therefore, feeding a portion of the hydrogen into the methanation reaction inlet while bypassing the steam reforming reactor has the effect of suppressing thermal degradation of the catalyst used for the steam reforming reaction.

TABLE 20

| | Temperature | Pressure | Flow rate [mol/s] | | | | | |
|---|---|---|---|---|---|---|---|---|
| | °C | MPa | H₂ | CO | CO₂ | H₂O | CH₄ | C₂H₅OH |
| Inlet of raw | 25 | 0.8 | 1.000 | 0.000 | 0.000 | 2.000 | 0.000 | 1.000 |
| material gas | | | | | | | | |
| conditioning | | | | | | | | |
| unit | | | | | | | | |
| Inlet of | 300 | 0.8 | 1.000 | 0.000 | 0.000 | 2.000 | 0.000 | 1.000 |
| steam | | | | | | | | |
| reforming | | | | | | | | |
| reactor | | | | | | | | |
| Outlet of | 568 | 0.8 | 1.204 | 0.099 | 0.477 | 1.947 | 1.424 | 0.000 |
| steam | | | | | | | | |
| reforming | | | | | | | | |
| reactor | | | | | | | | |
| Inlet of first | 256 | 0.8 | 2.204 | 0.099 | 0.477 | 1.947 | 1.424 | 0.000 |
| methanation | | | | | | | | |
| reactor | | | | | | | | |
| Outlet of | 506 | 0.8 | 1.036 | 0.022 | 0.242 | 2.493 | 1.735 | 0.000 |
| first | | | | | | | | |
| methanation | | | | | | | | |
| reactor | | | | | | | | |
| Inlet of | 250 | 0.8 | 1.036 | 0.022 | 0.242 | 2.493 | 1.735 | 0.000 |
| second | | | | | | | | |
| methanation | | | | | | | | |
| reactor | | | | | | | | |
| Outlet of | 250 | 0.8 | 0.072 | 0.000 | 0.018 | 2.964 | 1.982 | 0.000 |
| second | | | | | | | | |
| methanation | | | | | | | | |
| reactor | | | | | | | | |
| Fuel gas | 25 | 0.8 | 0.072 | 0.000 | 0.018 | 0.000 | 1.982 | 0.000 |
| (after | | | | | | | | |
| desiccation) | | | | | | | | |

### Example 13

The present example shows a calculation result of a fuel gas production process in the form shown in the block flow diagram of FIG. 5. The process flow is shown in FIG. 10.

In the raw material gas conditioning process, ethanol, hydrogen, and water are respectively supplied at flow rates of 1 mol/s, 2.0 mol/s, and 2.0 mol/s, at a temperature of 25 °C and a pressure of 0.8 MPa. In the raw material gas conditioning process, these components are heated and mixed, and the mixture is mixed with a recycle gas from the outlet of the steam reforming reactor 11 and heated to 300 °C to form a raw material gas, which is then fed into the steam reforming process.

In the steam reforming process, the raw material gas is fed into the steam reforming reactor 11, where a reaction proceed adiabatically. After the outlet gas from the steam reforming reactor 11 is cooled to 250 °C, one-third thereof is returned to the raw material gas conditioning process through the recycling compressor 5, and the remainder is fed into the first methanation reactor 21. The first methanation reactor 21 is an adiabatic reactor, and the outlet gas thereof is cooled to 250 °C in the heat exchanger 22 and then fed to the second methanation reactor 23. The second methanation reactor 23 is a heat exchanger reactor, where a methanation reaction proceeds at nearly 250 °C to an equilibrium composition of 250 °C. The outlet gas from the second methanation reactor 23 is cooled in the heat exchanger 24, and after separation of water and desiccation, a fuel gas that contains methane as the main component thereof is obtained.

Temperatures and flow rates at key positions in the process are shown in Table 21.

The composition of the obtained fuel gas (by volume after desiccation) contains 95.65% methane, 3.48% hydrogen, 0.87% carbon dioxide, and 2 ppm carbon monoxide. It is apparent that a fuel gas with high methane purity (high calorific value fuel gas) that can be used as city gas can be obtained by the method of the present invention. At the outlet of each reactor, the carbon activity is lower than 1, and carbon deposition is avoided in view of chemical equilibrium.

Comparing Example 10 and Example 13, the resulting fuel gas has the same composition. On the other hand, the temperature at the outlet of the steam reforming reactor is 596 °C in Example 10, and is 559 °C in Example 12, which is about 40 °C lower. The reason is that, due to the fact that a portion of the outlet gas at the steam reforming reaction unit has been returned to the inlet of the steam reforming reactor and recycled, dilution of the reaction gas suppressed the temperature rise. By returning a portion of the outlet gas at the steam reforming reaction unit to the inlet of the steam reforming reactor, thermal degradation of the catalyst used for the steam reforming reaction is suppressed.

In Example 13, at the stage where ethanol has been mixed with hydrogen and water vapor, the molar ratio of hydrogen/ethanol is 2.0 and the molar ratio of water vapor/ethanol is 2.0, but because the ethanol has been mixed with the gas returned from the outlet gas of the steam reforming process and then fed into the steam reforming process, the molar ratio of hydrogen/ethanol at the inlet of the steam reforming process is 2.736 and the molar ratio of water vapor/ethanol is 3.148, which are slightly different. Nevertheless, the value of {(hydrogen)+(ethanol)×6+(carbon monoxide)}/{(ethanol)×2+(carbon monoxide)+(carbon dioxide)} calculated based on amounts of substance (mol) of ethanol, hydrogen, carbon monoxide, and carbon dioxide supplied to the steam reforming process, is 4.0, and thus the raw material gases are blended in an appropriate composition, resulting in a fuel gas with high methane purity.

TABLE 21

| | Temperature | Pressure | Flow rate [mol/s] | | | | | |
|---|---|---|---|---|---|---|---|---|
| | °C | MPa | H₂ | CO | CO₂ | H₂O | CH₄ | C₂H₅OH |
| Inlet of raw | 25 | 0.8 | 2.000 | 0.000 | 0.000 | 2.000 | 0.000 | 1.000 |
| material gas | | | | | | | | |
| conditioning | | | | | | | | |
| unit | | | | | | | | |
| Inlet of steam | 300 | 0.8 | 2.736 | 0.032 | 0.160 | 3.148 | 0.808 | 1.000 |
| reforming | | | | | | | | |
| reactor | | | | | | | | |
| Outlet of | 559 | 0.8 | 2.209 | 0.097 | 0.479 | 3.444 | 2.423 | 0.000 |
| steam | | | | | | | | |
| reforming | | | | | | | | |
| reactor | | | | | | | | |
| Inlet of first | 250 | 0.8 | 1.473 | 0.065 | 0.320 | 2.296 | 1.616 | 0.000 |
| methanation | | | | | | | | |
| reactor | | | | | | | | |
| Outlet of first | 440 | 0.8 | 0.624 | 0.005 | 0.153 | 2.690 | 1.843 | 0.000 |
| methanation | | | | | | | | |
| reactor | | | | | | | | |
| Inlet of second | 250 | 0.8 | 0.624 | 0.005 | 0.153 | 2.690 | 1.843 | 0.000 |
| methanation | | | | | | | | |
| reactor | | | | | | | | |
| Outlet of | 250 | 0.8 | 0.072 | 0.000 | 0.018 | 2.964 | 1.982 | 0.000 |
| second | | | | | | | | |
| methanation | | | | | | | | |
| reactor | | | | | | | | |
| Fuel gas | 25 | 0.8 | 0.072 | 0.000 | 0.018 | 0.000 | 1.982 | 0.000 |
| (after | | | | | | | | |
| desiccation) | | | | | | | | |

### Example 14

The present example shows a calculation result of a fuel gas production process shown in the block flow diagram of FIG. 2, in which the raw material supplied to the steam reforming process further contains carbon dioxide. Note that as in Example 13, a configuration was employed in which a portion of the outlet gas at the steam reforming reaction unit is returned to the inlet of the steam reforming reactor.

In the raw material gas conditioning process, ethanol, hydrogen, water, and carbon dioxide are respectively supplied at flow rates of 1 mol/s, 6.0 mol/s, and 2.0 mol/s, and 1.0 mol/s, at a temperature of 25 °C and a pressure of 0.8 MPa. In the raw material gas conditioning process, these components are heated and mixed, and the mixture is mixed with a recycle gas from the outlet of the steam reforming reactor 11 and heated to 300 °C to form a raw material gas, which is then fed into the steam reforming process.

In the steam reforming process, the raw material gas is fed into the steam reforming reactor 11, where a reaction proceed adiabatically. After the outlet gas from the steam reforming reactor 11 is cooled to 250 °C, one-third thereof is returned to the raw material gas conditioning process through the recycling compressor 5, and the remainder is fed into the first methanation reactor 21. The first methanation reactor 21 is an adiabatic reactor, and the outlet gas thereof is cooled to 250 °C in the heat exchanger 22 and then fed to the second methanation reactor 23. The second methanation reactor 23 is a heat exchanger reactor, where a methanation reaction proceeds at nearly 250 °C to an equilibrium composition of 250 °C. The outlet gas from the second methanation reactor 23 is cooled in the heat exchanger 24, and after separation of water and desiccation, a fuel gas that contains methane as the main component thereof is obtained.

Temperatures and flow rates at key positions in the process are shown in Table 22.

The composition of the obtained fuel gas (by volume after desiccation) contains 95.35% methane, 3.72% hydrogen, 0.93% carbon dioxide, and 2 ppm carbon monoxide. It is apparent that a fuel gas with high methane purity (high calorific value fuel gas) that can be used as city gas can be obtained by the method of the present invention. At the outlet of each reactor, the carbon activity is lower than 1, and carbon deposition is avoided in chemical equilibrium.

In the present example, since the raw material gas is prepared by mixing ethanol with hydrogen, water vapor, and carbon dioxide, the molar ratio of hydrogen/ethanol is 6.0 at the raw material gas preparation stage, and the mixture is further mixed with the gas returned from the outlet gas of the steam reforming process and then fed into the steam reforming process, the molar ratio of hydrogen/ethanol at the inlet of the steam reforming process is 7.581, and the molar ratio of water vapor/ethanol is 3.768, the molar ratios being slightly higher. Nevertheless, the value of {(hydrogen)+(ethanol)×6+(carbon monoxide)}/{(ethanol)×2+(carbon monoxide) 10+(carbon dioxide)} calculated based on amounts of substance (mol) of ethanol, hydrogen, carbon monoxide, and carbon dioxide supplied to the steam reforming process is 4.0, and thus the raw material gases are blended in an appropriate composition, resulting in a fuel gas with high methane purity.

TABLE 22

| | Temperature | Pressure | Flow rate [mol/s] | | | | | |
|---|---|---|---|---|---|---|---|---|
| | °C | MPa | H₂ | CO | CO₂ | H₂O | CH₄ | C₂H₅OH |
| Inlet of raw | 25 | 0.8 | 6.000 | 0.000 | 1.000 | 2.000 | 0.000 | 1.000 |
| material gas | | | | | | | | |
| conditioning | | | | | | | | |
| unit | | | | | | | | |
| Inlet of | 300 | 0.8 | 7.581 | 0.117 | 1.307 | 3.768 | 1.075 | 1.000 |
| steam | | | | | | | | |
| reforming | | | | | | | | |
| reactor | | | | | | | | |
| Outlet of | 609 | 0.8 | 4.742 | 0.352 | 0.922 | 5.304 | 3.226 | 0.000 |
| steam | | | | | | | | |
| reforming | | | | | | | | |
| reactor | | | | | | | | |
| Inlet of first | 250 | 0.8 | 3.162 | 0.234 | 0.615 | 3.536 | 2.151 | 0.000 |
| methanation | | | | | | | | |
| reactor | | | | | | | | |
| Outlet of | 494 | 0.8 | 1.533 | 0.026 | 0.363 | 4.247 | 2.610 | 0.000 |
| first | | | | | | | | |
| methanation | | | | | | | | |
| reactor | | | | | | | | |
| Inlet of | 250 | 0.8 | 1.533 | 0.026 | 0.363 | 4.247 | 2.610 | 0.000 |
| second | | | | | | | | |
| methanation | | | | | | | | |
| reactor | | | | | | | | |
| Outlet of | 250 | 0.8 | 0.116 | 0.000 | 0.029 | 4.942 | 2.971 | 0.000 |
| second | | | | | | | | |
| methanation | | | | | | | | |
| reactor | | | | | | | | |
| Fuel gas | 25 | 0.8 | 0.116 | 0.000 | 0.029 | 0.000 | 2.971 | 0.000 |
| (after | | | | | | | | |
| desiccation) | | | | | | | | |

### OTHER EMBODIMENTS

Finally, other embodiments of the method for producing a high calorific value fuel gas according to the present invention will be described. Note that the configurations disclosed in the following embodiments can be applied in combination with the configurations disclosed in other embodiments as long as no contradiction arises.

In the embodiments of Examples 3 to 14, an example has been specifically described in which a steam reforming reactor used in the steam reforming process is an adiabatic reactor. However, the reactor used in the steam reforming process according to the present invention is not particularly limited and may be a heat exchanger reactor.

In the embodiments of Examples 10 to 14, an example has been specifically described in which the methanation reactor used in the methanation process constituted by the first methanation reactor 21, which is an adiabatic reactor, and the second methanation reactor 23, which is a heat exchanger reactor. However, the reactor used in the methanation process is not particularly limited as long as the outlet temperature of the catalyst layer in the final stage of the methanation reactor is 230 °C or more and 330 °C or less, and may consist of two or more adiabatic reactors connected to each other, or two or more heat exchanger reactors connected to each other.

In the embodiments of Examples 10 to 14, an example has been specifically described in which the steam reforming reactor and the methanation reactor used in the methanation process are housed in separate reactors, each reactor is provided with at least one heat exchanger, and the gas from the steam reforming reactor is cooled through the heat exchanger and then inserted into the methanation reactor. However, the steam reforming reactor and the methanation reactor may be formed in one piece, and for example, by filling a single reactor with the same or different catalysts and designing the reactor without a heat exchanger in the vicinity of the inlet or designing a smaller heat exchange function and providing it in the vicinity of the inlet instead of the vicinity of the outlet, the reaction may be allowed to proceed under adiabatic or almost adiabatic conditions in the first half of the reactor, while the reaction may be allowed to proceed under isothermal or almost isothermal conditions in the second half of the reactor. In this case, it is also possible to design such that the raw material gas is introduced into the steam reforming reactor at 300 °C, and in the first half, the reaction proceeds adiabatically so that the peak temperature of the catalyst layer is about 400 °C, for example, and in the second half, the gas produced by the steam reforming reaction is gradually cooled so that a methanation reaction proceeds, thereby the gas leaving the reactor at a temperature of 230 °C or more and 330 °C or less. In this case, the steam reforming process is performed in the first half of the reactor, and the methanation process is performed in the second half of the reactor.

In addition, the present invention does not exclude the combined use of conventionally known means for improving the methanation rate. For example, it is possible to employ a configuration for cooling the reaction gas in an intermediate stage of the multi-stage reaction to condense and separate some water vapor. Even in such a case, by using the method according to the present invention, it is possible to reduce the hydrogen and carbon dioxide concentrations in the fuel gas without removing water vapor extremely.

Also, the present invention does not exclude the combined use of known means for improving methane purity. For example, by adding a small amount of oxygen to the fuel gas after a methanation reaction and conducting a selective oxidation reaction to selectively oxidize only hydrogen, it is possible to reduce hydrogen from the fuel gas after the methanation reaction to obtain fuel gas with higher methane purity.

In the embodiment of Example 14, the raw material gas has a configuration in which it contains carbon dioxide, but the present invention is not limited to such an example. The raw material gas may contain carbon monoxide, or may contain carbon monoxide and carbon dioxide.

The configuration disclosed in the above embodiments (including other embodiments, the same hereinafter) can be applied in combination with the configuration disclosed in other embodiments, as long as no contradiction arises. The embodiments disclosed in the present specification are examples, and the present invention is not limited thereto, and modifications may be made as appropriate within the scope that does not depart from the purpose of the present invention.

### INDUSTRIAL APPLICABILITY

The present invention is usable as a method for producing a fuel gas to be supplied as city gas, for example.

### DESCRIPTION OF REFERENCE SIGNS

1: Steam reforming reaction unit
2: Methanation reaction unit
3: Raw material gas conditioning section
4: Heat exchanger
5: Recycling compressor
11: Steam reforming reactor
12: Heat exchanger
21: First methanation reactor
22: Heat exchanger
23: Second methanation reactor
24: Heat exchanger

1. A method for producing a high calorific value fuel gas comprising:
a steam reforming process comprising bringing ethanol into contact with a catalyst containing ruthenium or nickel in the presence of hydrogen in an amount such that a molar ratio of hydrogen/ethanol is 1.0 or more and 10 or less, and water vapor in an amount such that a molar ratio of water vapor/ethanol is 1.2 or more and 5 or less, under a condition where an inlet temperature of the catalyst is 200 °C or more and less than 400 °C; and
a methanation process comprising bringing a gas obtained in the steam reforming process into contact with a catalyst containing ruthenium or nickel under a condition where an outlet temperature of the catalyst is 230 °C or more and 330 °C or less.

2. The method for producing a high calorific value fuel gas according to claim 1,
wherein a raw material gas to be subjected to the steam reforming process further contains at least one of carbon monoxide and carbon dioxide.

3. The method for producing a high calorific value fuel gas according to claim 2,
wherein an amount of hydrogen to be subjected to the steam reforming process has a value such that a value of {(hydrogen)+(ethanol)×6+(carbon monoxide)}/{(ethanol)×2+(carbon monoxide)+(carbon dioxide)} calculated based on an amount of substance (mol) is 3.9 or more and 4.1 or less.

4. The method for producing a high calorific value fuel gas according to claim 1, further comprising:
a process of comprising conditioning a raw material gas by adding hydrogen and water vapor to ethanol so that the molar ratio of hydrogen/ethanol is 1.8 or more and 2.2 or less and the molar ratio of water vapor/ethanol is 1.2 or more and 4 or less, and
wherein the raw material gas is subjected to the steam reforming process.

5. The method for producing a high calorific value fuel gas according to claim 1, further comprising:
a process comprising conditioning a raw material gas by adding hydrogen and water vapor to ethanol so that the molar ratio of hydrogen/ethanol is 1.0 or more and less than 1.8 and the molar ratio of water vapor/ethanol is 2 or more and 4 or less, and
wherein the raw material gas is subjected to the steam reforming process, hydrogen is added to a gas obtained in the steam reforming process, and a resulting gas is subjected to the methanation process.

6. The method for producing a high calorific value fuel gas according to claim 4,
wherein the raw material gas conditioned by adding hydrogen and water vapor to ethanol is mixed with a portion of a gas obtained in the steam reforming process, and is then subjected to the steam reforming process.
